(19) United States
(12) Patent Application Publication
(10) Pub. No.: US 2009/0187108 A1
(43) Pub. Date: Jul. 23, 2009

(54) SYSTEMS AND METHODS FOR ANALYSIS AND TREATMENT OF A BODY LUMEN

(75) Inventors: Jing Tang, Arlington, MA (US); S. Eric Ryan, Hopkinton, MA (US)

Correspondence Address: MILLS & ONELLO LLP, ELEVEN BEACON STREET, SUITE 605, BOSTON, MA, 02108, US

(73) Assignee: CORNOVA, INC., Burlington, MA (US)

(21) Appl. No.: 12/350,870

(22) Filed: Jan. 8, 2009

Related U.S. Application Data
(63) Continuation-in-part of application No. 11/537,258, filed on Sep. 29, 2006.
(60) Provisional application No. 61/019,626, filed on Jan. 8, 2008.
(60) Provisional application No. 61/025,514, filed on Feb. 1, 2008.
(60) Provisional application No. 61/082,721, filed on Jul. 22, 2008.

(51) Int. Cl. A61B 6/08; A61N 5/06

(52) U.S. Cl. 600/475; 607/88; 600/477

(57) ABSTRACT

A catheter is provided for placement within a body lumen, the catheter including a flexible conduit that is elongated along a longitudinal axis, the flexible conduit having a proximal end and a distal end. The catheter further includes at least one delivery waveguide and at least one collection waveguide positioned along the flexible conduit, the at least one delivery waveguide and the at least one collection waveguide constructed and arranged to transmit radiation at a wavelength in a range of about 250 to 2500 nanometers. The catheter further includes a flexible, expandable first surface encircling surrounding a segment of the conduit, a transmission output of the at least one delivery waveguide and a transmission input of the at least one collection waveguide located within the flexible, expandable first surface, and the distal end of at least one of the at least one delivery waveguide and the at least one collection waveguide tethered to the flexible, expandable first surface radially translatable with respect to the flexible, expandable first surface, the at least one transmission input located between a portion of the flexible, expandable first surface and a portion of the second surface.

### RELATED APPLICATIONS

This application is a continuation-in-part of U.S. patent application Ser. No. 11/537,258, filed on Sep. 29, 2006, the entire contents of which is herein incorporated by reference. This application claims the benefit of U.S. Patent Application No. 61/019,626, filed Jan. 8, 2008, U.S. Patent Application No. 61/025,514, filed Feb. 1, 2008, and U.S. Patent Application No. 61/082,721 filed Jul. 22, 2008, the entire contents of each of which is herein incorporated by reference. This application is related to U.S. patent application Ser. No. 11/834,096, filed on Aug. 6, 2007, published as U.S. Patent Application Publication No. 2007/0270717 A1, the entire contents of which is herein incorporated by reference. This application is related to U.S. Ser. No. ______, filed on or around the filing date of the present application, entitled “Shaped Fiber Ends and Methods of Making Same,” by Jing Tang, the contents of which is incorporated herein in their entirety by reference.

### BACKGROUND OF THE INVENTION

1. Field of the Invention

Embodiments of the present invention are directed to systems and methods for the analysis and treatment of a lumen. More particularly, the present invention relates to a balloon catheter system that is used to perform methods of analysis and angioplasty of endovascular lesions.

2. Description of the Related Art

With the continual expansion of minimally-invasive procedures in medicine, one procedure that has been highlighted in recent years has been percutaneous transluminal angioplasty, or “PTA”. The most prevalent use of this procedure is in the coronary arteries, which is more specifically called a percutaneous coronary transluminal angioplasty, or “PTCA”. These procedures utilize a flexible catheter with an inflation lumen to expand, under relatively high pressure, a balloon at the distal end of the catheter to expand a stenotic lesion.

The PTA and PTCA procedures are now commonly used in conjunction with expandable tubular structures known as stents, and an angioplasty balloon is often used to expand and permanently place the stent within the lumen. An angioplasty balloon utilized with a stent is referred to as a stent delivery system. Conventional stents have been shown to be more effective than angioplasty alone in maintaining patency in most types of lesions and also reducing other near-term endovascular events. A risk with a conventional stent, however, is the reduction in efficacy of the stent due to the growth of the tissues surrounding the stent which can again result in the stenosis of the lumen, often referred to as restenosis. In recent years, new stents that are coated with pharmaceutical agents, often in combination with a polymer, have been introduced and shown to significantly reduce the rate of restenosis. These coated stents are generally referred to as drug-eluting stents, though some coated stents have a passive coating instead of an active pharmaceutical agent.

With the advent of these advanced technologies for PTA and PTCA, there has been a substantial amount of clinical and pathology literature published about the pathophysiologic or morphologic factors within an endovascular lesion that contribute to its restenosis or other acute events such as thrombosis. These features include, but are not limited to, collagen content, lipid content, calcium content, inflammatory factors, and the relative positioning of these features within the plaque. Several studies have been provided showing the promise of identifying the above factors through the use of visible and/or near infrared spectroscopy, i.e., across wavelengths ranging between about 250 to 2500 nm, including those studies referenced in U.S. Publication No. US2004/0111016A1 by Casscells, III et al., U.S. Publication No. US2004/0077950A1 by Marshik-Geurts et al., U.S. Pat. No. 5,304,173 by Kittrell et al., and U.S. Pat. No. 6,095,982 by Richards-Kortum, et al., the contents of each of which are herein incorporated by reference. However, there are very few, if any, highly safe and commercially viable applications making use of this spectroscopic data for combining diagnosis and treatment in a PTA or PTCA procedure.

In addition, dynamic and optimal control over the expansion of the balloon during angioplasty procedures is very limited, including during pre-dilation of the vasculature prior to stent delivery, dilation during stent delivery, and post-dilation after delivery of a stent. Underexpansion of an angioplasty balloon may require deployment of an additional catheter and stent in order to complete the desired treatment and/or ensure that an underexpanded stent is not blocking blood flow through a vessel and can thus complicate a procedure, resulting in increased risks, and added expense. Information about the apposition and expansion of the balloon against the vessel walls during these procedures could therefore be highly useful for mitigating these risks.

Typical technologies used for monitoring angioplasty and stenting procedures include angiography by fluoroscopy, which supplies an X-ray image of the blood flow within a lumen. However, this technology has a very limited resolution of about 300 micrometers. As a result, many angioplasty and stenting procedures overexpand the lumen, which can result in unnecessary trauma and damage to the lumen wall, complicating post-deployment recovery, and increasing the likelihood of re-closure of the lumen (restenosis).

Angioscope technology is also generally used for identifying a stenosis, but provides no information about the endovascular wall of the plaque. Some important diseases located on non- or minor stenosis regions, such as a vulnerable plaque which is fatal to a patient life, are often missed. Moreover, radiation delivered by an angiography procedure can have negative side-effects. Other technologies, such as intravascular ultrasound, require expensive additional catheters and potentially dangerous additional procedures that can cause more harm than good and still not supply sufficient information about the plaque to be beneficial. There are currently needs for physicians to gain this useful information about the lumen wall, including accurately locating diseased tissue for purposes of conducting angioplasty procedures in an accurate, cost-effective, and efficient manner that presents a reasonable risk profile for the patient.

Conventional balloon catheters are not generally used for purposes other than for performing traditional angiosplasty procedures including pre-dilation of the vasculature prior to stent delivery, stent delivery, and post-stent delivery dilation. A capability that is not presently available and would be highly valuable before, during, and after such procedures would be the ability to assess the optimal type of stent and/or stent coating, if any, to deploy. The availability of the aforementioned pathophysiologic or morphologic factors could be used to help such assessments.

Furthermore, the level and uniformity of expansion of balloons during such procedures is only roughly determined, e.g., with use of an angiogram and a balloon expansion estimation charts, and is often unnecessarily exceeded in order to avoid issues associated with underexpansion as previously discussed. Overexpansion, however, carries its own risks including, for example, rupture of a lesion or excessive damage to a weakened vessel wall. For these reasons, stent deployment may be avoided altogether and substituted with less risky but less effective procedures.

Prior use of optical fibers within an angioplasty catheter permit functions such as visualization to occur, but limited information from such techniques can be obtained. Conventional balloon catheters generally have no capacity to collect any information beyond the surface of the endovascular wall that can be critical to proper diagnosis and treatment of diseased vessels. While lower-pressure balloon catheters are available to occlude the blood flow proximal to the optical analysis window of a catheter, no lumen expansion is performed and no analysis can be performed within the balloon itself. Other systems support the use of optical feedback within a balloon catheter to atraumatically minimize the blood path between the balloon catheter and the endovascular wall. However, these systems likewise provide no ability to perform a complete optical analysis of the lumen wall.

### SUMMARY OF THE INVENTION

The systems and methods described in the present specification provide physicians performing a lumen-expansion procedure with very useful information about the lumen wall without any significant increase in their procedure time or cost, and with little to no additional risk to the patient. Included are a number of implementations of distal fiber-optic configurations to optimally facilitate analysis of the lumen wall and angioplasty balloon characteristics. These implementations also provide manufacturability and relatively low-cost production required for a disposable medical device.

In an embodiment, the distal fiber optical configuration distributes at least one delivery waveguide and at least one collection waveguide with distal ends arranged such that, upon expansion of the balloon catheter in a body lumen, the distal waveguide ends are positioned proximate to the perimeter of the catheter's treatment end with little or no media fluid or bodily fluid positioned between the distal waveguide ends and the lumen wall. In an embodiment, the apparatus includes an inside balloon and an outside covering surrounding the inside balloon. In an embodiment, as the inside balloon is expanded with fluid media, the inside balloon positions the distal waveguide ends proximate to the outside covering and a lumen wall. In an embodiment, the outside covering is filled with fluid media so as to operate as a lumen expanding balloon.

In an embodiment, the apparatus consists of a single balloon to which the waveguide ends are held against such that they remain proximate to the balloon's wall during expansion with fluid media.

In an embodiment of the invention, the delivery and collection ends of fibers of the optical configuration are adapted for near-field, wide scope use. The adaptation is particularly advantageous where the delivery and/or collection ends are to be positioned closely to targeted tissue and/or blood during deployment as in various embodiments described herein. In an embodiment, at least one delivery and/or a collection end is manufactured using a controlled etching process. In an embodiment, fiber tips are formed through emersion in a liquefied etchant such as, for example, hydrofluoric acid over a pre-determined period of time.

In one embodiment, optical analysis of the plaque is performed within the same catheter utilized for angioplasty during a PTA or PTCA procedure. This optical analysis could include, but not limited to, Raman spectroscopy, infrared spectroscopy, fluorescence spectroscopy, optical coherence reflectometery, optical coherence tomography, but most preferably diffuse-reflective, near-infrared spectroscopy. The embodiment provides optical analysis, and thus the pathophysiologic or morphologic features diagnosis, of a plaque during an angioplasty procedure without any significant additional cost, risk, or work for the physician. With access to this information, a physician could potentially choose from a selection of drug-eluting stents with different doses or agents, or even select a stent without a drug if indicated. During typical angioplasty procedures performed on a patient, including pre-dilation of a lumen, stent delivery, and/or post-dilation, a physician could learn more about the general status of the patient's vasculature which can guide systemic therapies. New emerging technologies such as bioabsorbable stents could be enabled by the embodiments of the invention to optimize their use in the correct type of lesion.

In addition to obtaining information useful to diagnosis, an embodiment of the invention obtains information about the level of expansion of the balloon within the lumen. In an embodiment, information is collected about the amount of blood between the balloon wall and a lumen so as to determine if and when the balloon is fully apposed to the lumen wall and/or to help diagnose and locate pathophysiologic or morphologic factors. Information about the balloon with respect to the lumen can be used to control the balloon's expansion so that it does not under-expand or over-expand during treatment. In certain circumstances, a lesion and/or deposit can cause an angioplasty balloon to become mal-apposed upon expansion. In an embodiment of the invention, levels of blood are measured about the balloon perimeter to help diagnose hard lesions.

In an aspect of the invention, a catheter is provided for placement within a body lumen, the catheter including a flexible conduit that is elongated along a longitudinal axis, the flexible conduit having a proximal end and a distal end. The catheter further includes at least one delivery waveguide and at least one collection waveguide positioned along the flexible conduit, the at least one delivery waveguide and the at least one collection waveguide constructed and arranged to transmit radiation at a wavelength in a range of about 250 to 2500 nanometers. The catheter further includes a flexible, expandable first surface surrounding a segment of the conduit, the transmission output and a transmission input located within the flexible, expandable first surface, and a second surface radially translatable with respect to the flexible, expandable first surface, the at least one transmission input located between a portion of the flexible, expandable first surface and a portion of the second surface.

In an embodiment, at least one of the first surface and the second surface forms a surface of a lumen-expanding balloon.

In an embodiment, the lumen-expanding balloon is an angioplasty balloon.

In an embodiment, a stent is mounted over the first surface.

In an embodiment, the at least one of the delivery and collection waveguides include at least one optical fiber and wherein the longitudinal axis of a tip of the at least one optical fiber is arranged to be substantially parallel with the first surface.

In an embodiment, the at least one waveguide includes at least one fiber optic having a recess formed out of the distal end of the at least one fiber optic so as to allow the transmission of radiation in a direction transverse to the longitudinal axis of the tip. In an embodiment, the recess includes a vertex located within the core of the at least one fiber optic. In an embodiment, the recess is at least one of elliptically shaped and conically shaped. In an embodiment, at least a portion of the recess is filled with a reflective material, light diffusing material and/or light blocking material. In an embodiment, an air gap is formed between the recess and the reflective material, light diffusing material, and/or light blocking material. In an embodiment, the at least one fiber optic is arranged to circumferentially emit or collect radiation around approximately 90 degrees or more of the end of the at least one fiber optic. In an embodiment, the at least one fiber optic includes graded-index core.

In an embodiment, the catheter includes a first conduit for directing inflation media to the interior of the flexible, expandable first surface.

In an embodiment, the catheter includes a second conduit for directing inflation media between the flexible expandable first surface and the second surface.

In an embodiment, the first conduit and the second conduit are arranged to initially direct more inflation media to the interior of the flexible, expandable first surface in which inflation media is directed to the area between the flexible, expandable first surface and the second surface.

In an embodiment, the first conduit includes a greater volumetric capacity for transferring fluid than the second conduit.

In an embodiment, first conduit is in direct fluid communication to each of the inside of the flexible, expandable first surface and the area between the flexible, expandable first surface and the second surface.

In an embodiment, the second surface includes a reflective surface.

In an embodiment, the reflective surface forms a circumferential band around the flexible conduit.

In an embodiment, the reflective surface includes at least one of a gold-colored and silver-colored coating.

In an embodiment, the coating includes paint.

In an embodiment, the reflective surface is applied to the catheter by an ion-assisted deposition method.

In an embodiment, the reflective surface is concave with respect to the at least one delivery waveguide and the at least one collection waveguide.

In an embodiment, the reflective surface includes a translucent gap through which light radiation can pass between a transmission input or output located outside the periphery of the reflective surface and an area located within the periphery of the reflective surface.

In an embodiment, one or more additional surfaces translatable with respect to the flexible, expandable first surface and wherein one or more additional transmission outputs or inputs are located between a portion of the flexible expandable first surface and portions of the one or more additional surfaces.

In an embodiment, the additional surfaces each include a reflective surface.

In an embodiment, each of the additional surfaces includes an eyelet attached to the first surface, wherein at least one waveguide passes through an eyelet.

In an embodiment, each of the additional surfaces includes a reflective element.

In an embodiment, each of the additional surfaces is attached to at least one of the at least one delivery waveguide and at least one collection waveguide and wherein each of the additional surfaces is attached to the second surface.

In an embodiment, the first surface and the second surface form at least one pocket which holds at least one of the at least one delivery waveguide and the at least one collection waveguide.

In an embodiment, the first surface and the second surface are arranged so as to hold the tip of the at least one delivery waveguide and the at least one collection waveguide at a predetermined distance from the first surface when the first surface is fully expanded.

In an embodiment, the at least one delivery waveguide and at least one collection waveguide comprise no more than 6 waveguides. In an embodiment, the at least one delivery waveguide and at least one collection waveguide comprise 4 waveguides. In an embodiment, at least one of the delivery and collection waveguides has a maximum outer diameter of less than about 80 microns.

In an embodiment, the transmission output of the at least one delivery waveguide and the transmission input of the at least one collection waveguide are arranged to facilitate collection of radiation emitted from tissue of a predetermined scope and depth from the flexible, expandable first surface. In an embodiment, the transmission output of the at least one delivery waveguide and the transmission input of the at least one collection waveguide are spaced apart at a predetermined distance to facilitate the collection of radiation emitted from tissue of a predetermined scope and depth from the flexible, expandable first surface. In an embodiment, the predetermined distance includes a longitudinal component. In an embodiment, the predetermined distance includes a circumferential component.

In an embodiment, the catheter further includes a waveguide having a transmission input or transmission output that is contiguously retained against the flexible conduit.

In an embodiment, the transmission output or transmission input that is contiguously retained against the flexible conduit is arranged to deliver or collect radiation transmitted to or from a waveguide retained against the first surface.

In an embodiment, the arrangement to deliver or collect radiation transmitted to or from a waveguide retained against the first surface is configured to provide information including the uniformity of expansion of the flexible, expandable first surface.

In an embodiment, the at least one waveguide extending along the flexible conduit is slidably movable along the longitudinal axis of the flexible conduit.

In an embodiment, the second surface includes a plurality of circumferential reflective bands distributed about the longitudinal axis of the flexible conduit.

In an embodiment, the plurality of circumferential reflective bands include two bands, one of the two bands positioned at a proximate end of the first surface and one of the two bands positioned at a distal end of the first surface so as to form a translucent region between the two reflective bands.

In an embodiment, the catheter includes a slidably movable handle located at the proximate end of the flexible conduit, the slidably movable handle connected to the at least one slidably movable waveguide so as to allow for slidably moving the at least one slidably movable waveguide.

In an embodiment, the slidably movable handle includes a mechanical locking mechanism for positioning the slidably movable waveguides at predetermined longitudinal positions along the first surface.

In an embodiment, each of the at least one slidably movable waveguide is retained in a sleeve within which the at least one slidably movable waveguide can slide. In an embodiment, sleeve is constructed of a translucent material.

In an aspect of the invention, a system for probing and treating a body lumen is provided that includes a flexible conduit that is elongated along a longitudinal axis suitable for insertion into a body lumen, the conduit having a proximal end and a distal end. The flexible conduit is integrated with at least one delivery waveguide and at least one collection waveguide. At least one radiation source is connected to a transmission input of the at least one least one delivery waveguide. The radiation source is constructed and arranged to provide radiation at a wavelength in a range of about 250 to 2500 nanometers. At least one optical detector is connected to a transmission output of the at least one collection waveguide. The system includes a controller. A flexible, expandable first surface encircles a segment of the conduit wherein the transmission output of the at least one delivery waveguide and the transmission input of the at least one collection waveguide are located within the flexible, expandable first surface. The at least one transmission input is movably coupled to the first surface.

In an embodiment, the transmission output of the at least one collection waveguide is connected to a spectrometer. In an embodiment, the spectrometer is constructed and arranged to scan radiation and perform spectroscopy at the wavelength in the range of about 250 nm to 2500 nm.

In an embodiment, the spectrometer and controller are configured to perform one or more spectroscopic methods including at least one of fluorescence, light scatter, optical coherence reflectometry, optical coherence tomography, speckle correlometry, Raman, and diffuse reflectance spectroscopy.

In an embodiment, the spectrometer is constructed and arranged to scan radiation and perform spectroscopy at a wavelength within the range of about 750 nm to 2500 nm. In an embodiment, the spectrometer is constructed and arranged to scan radiation and perform spectroscopy using one or more ranges of wavelengths.

In an embodiment, the spectrometer is constructed and arranged to scan radiation and perform spectroscopy using one or more discrete wavelengths.

In an embodiment, the system is configured to identify one or more characteristics of targeted tissue including at least one of: presence of chemical components, tissue morphological structures, water content, blood content, temperature, pH, and color. In an embodiment, the one or more characteristics includes the presence of a gap between the first surface and the targeted tissue.

In an embodiment, the system is configured for determining the level of apposition of the first surface against adjacent tissue based on the identification of blood adjacent the first surface.

In an embodiment, the one or more characteristics includes a gap with a distance between the first surface and the targeted tissue.

In an embodiment, the system is configured for controlling the level of expansion of the first surface based on the distance of the first surface in relation to the targeted tissue.

In an embodiment, the system is configured for the identification of blood by inducing and detecting fluorescence. In an embodiment, the system includes a dichroic filter arranged to separate radiation of wavelengths selected for delivery and radiation of wavelengths selected for collection.

In an embodiment, the radiation source is configured to supply radiation including a wavelength of 450 nanometers and wherein the optical detector is configured and arranged to selectively detect radiation including a wavelength of 520 nanometers.

In an embodiment, the radiation source is configured to supply radiation of one or more wavelengths including about 532 nanometers, 407 nanometers, and between about 800 and 1000 nanometers.

In an embodiment, the one or more wavelengths consist of two wavelengths including at least one of about 532 nanometers.

In an embodiment, the system is programmed to calculate a ratio of absorbance data from the collection of the one or more wavelengths and compare the ratio with predetermined data including relationships between pre-calculated ratios of corresponding absorbance data in relation to known blood depths proximate a vessel wall.

In an embodiment, the system includes an indicator of signal intensity to an operator in relation to the identification of one or more characteristics of targeted tissue.

In an embodiment, the system is configured to discriminate between tissue characteristics and non-relevant artifacts including elements of the catheter and other elements artificially introduced into the body lumen.

In an embodiment, the system is configured to identify whether the first surface is fully expanded.

In an embodiment, the system is configured and programmed to identify whether the first surface is fully expanded by analyzing the characteristics of signals substantially transmitted within the circumference of the first surface.

In an embodiment, the signals substantially transmitted within the circumference of the first surface are directed between a plurality of transmission inputs and outputs positioned along the circumference of the first surface.

In an embodiment, the signals substantially transmitted within the circumference of the first surface are directed between one or more transmission inputs and outputs positioned along the circumference of the first surface and one or more transmission inputs or outputs positioned contiguously along the flexible conduit.

In an embodiment, the system is programmed to analyze and compare the signals for the amount of balloon inflation media present across the path of the signals.

In an embodiment, the analyzing and comparing signals for the amount of balloon inflation media detected includes comparing signals transmitted between different pairs of transmission inputs and outputs.

In an embodiment, the programming to analyze and compare the signals compares and distinguishes signals traveling across circumferential regions about the flexible conduit.

In an embodiment, the circumferential regions comprise quadrants about the flexible conduit.

In an aspect of the invention, a method for treating a body lumen is provided. The method includes the step of inserting into a body lumen a catheter. The catheter includes a flexible conduit with a flexible expandable surface encircling a segment of the conduit, at least one delivery waveguide and at least one collection waveguide. The delivery waveguide has a delivery output located within the flexible expandable surface and the collection waveguide has a collection input located within the flexible expandable surface. The method further includes the steps of maneuvering the conduit into a designated region of the body lumen designated for treatment or analysis, expanding the flexible expandable surface in the designated region of the body lumen while holding at least one collection input of at least one collection waveguide against the inside of the flexible expandable surface, and executing spectroscopic analysis of the designated region of the body lumen using radiation at a wavelength in the range of about 250 to 2500 nanometers. Radiation delivered to the designated region of the body lumen is supplied through the transmission output of the at least one delivery waveguide, the supplied radiation passing through the flexible expandable surface where it is incident on the designated region of the body lumen, and wherein radiation is returned through the flexible expandable surface to the transmission input of the at least one collection waveguide.

In an embodiment, the distal end of the at least one collection input is substantially parallel to the flexible expandable surface.

In an embodiment, executing spectroscopic analysis includes characterizing whether blood is passing between the catheter and a wall of the body lumen.

In an embodiment, characterizing whether blood is passing between the catheter and a wall of the body lumen occurs prior to the full expansion of the flexible expandable surface.

In an embodiment, characterizing whether blood is passing between the catheter and a wall of the body lumen occurs during the expansion of the flexible expandable surface.

In an embodiment, during the step of characterizing whether blood is passing between the catheter and a wall of the body lumen, an indicator relays a level of blood presence to an operator.

In an embodiment, characterizing whether blood is passing between the catheter and a wall of the body lumen is used to determine whether a stent that is positioned about the catheter is properly deployed. In an embodiment, determining whether a stent is properly deployed about the catheter includes determining whether the stent is mal-apposed.

In an embodiment, characterizing whether blood is passing between the catheter and a wall of the body lumen is performed by selectively supplying radiation including that of a wavelength of 450 nanometers and detecting fluorescence radiation including that of a wavelength of 520 nanometers.

In an embodiment, the spectrometer performs one or more spectroscopic methods including at least one of fluorescence, light scatter, optical coherence reflectometry, optical coherence tomography, speckle correlometry, Raman, and diffuse reflectance spectroscopy.

In an embodiment, the spectroscopy is performed at one or more wavelengths within the range of about 750 nm to 2500 nm.

In an embodiment, the spectroscopy is adapted to identify the presence of at least one of chemical components, tissue morphological structures, water content, blood content, temperature, pH, and color.

In an embodiment, the spectroscopy is used to perform a distance measurement between the first surface and the targeted tissue.

In an embodiment, the step of expanding the designated region of the body lumen includes expanding the designated region a predetermined amount based upon the distance measurement between the first surface and the targeted tissue.

In an embodiment, the spectroscopic analysis discriminates between tissue characteristics and non-relevant artifacts including elements of the catheter and other elements artificially introduced into the body lumen.

In an embodiment, executing spectroscopic analysis includes identifying whether the flexible expandable surface is fully expanded.

In an embodiment, executing spectroscopic analysis includes analyzing characteristics of signals transmitted substantially within the circumference of the flexible expandable surface.

In an embodiment, the signals are transmitted between one or more transmission inputs and outputs positioned along the circumference of the flexible expandable surface.

In an embodiment, the signals are transmitted between one or more transmission inputs and outputs positioned along the circumference of the flexible expandable surface and one or more transmission inputs or outputs positioned contiguously along the flexible conduit.

In an embodiment, analyzing characteristics of signals includes determining the presence and amount of balloon inflation media across the path of the signals.

In an embodiment, analyzing characteristics of signals further includes comparing the amount of balloon inflation media detected within signals transmitted between different pairs of transmission inputs and outputs.

In an aspect of the invention, a method is provided for forming a catheter for placement within a body lumen including the steps of providing a flexible conduit that is elongated along a longitudinal axis suitable for insertion into a body lumen. The flexible conduit includes a proximal end and a distal end. The method further includes the step of providing at least one delivery waveguide and at least one collection waveguide along the flexible conduit, the at least one delivery waveguide and the at least one collection waveguide constructed and arranged to transmit radiation at a wavelength in a range of about 250 to 2500 nanometers. The method further includes the steps of surrounding a segment of the conduit with a flexible, expandable first surface and providing a second surface that movably couples the radial movement of at least one of a transmission input of the at least one collection waveguide and a transmission output of the at least one delivery waveguide to the radial movement of the flexible, expandable first surface.

In an embodiment, at least one of the flexible, expandable first surface and second surface is an angioplasty balloon.

In an embodiment, a stent is mounted over the angioplasty balloon.

In an embodiment, the second surface includes a flexible, expandable covering over the flexible, expandable first surface.

In an embodiment, one or more conduits are provided for directing inflation media to an area inside the flexible, expandable first surface and to an area between the flexible, expandable first surface and the second surface.

In an embodiment, the one or more conduits are arranged to initially direct more inflation media to the inside of the flexible expandable first surface prior to directing inflation media to the area between the flexible, expandable first surface and the second surface.

In an embodiment, one of the one or more conduits is positioned in fluid communication between the inside of the flexible, expandable first surface and the area between the flexible, expandable first surface and the second surface.

In an embodiment, at least one of the at least one delivery waveguide and at least one collection waveguides is affixed to the flexible, expandable first surface by the second surface.

In an embodiment, the second surface is an adhesive.

In an embodiment, the second surface is formed as an eyelet on the flexible, expandable first surface, the one at least one delivery waveguide and at least one collection waveguides passing through the eyelet.

In an embodiment, the flexible, expandable first surface and the second surface are formed as a pocket wherein the at least one collection waveguide are held.

In an embodiment, the pocket is formed while the at least one collection waveguide is placed between the flexible, expandable first surface and the second surface.

In an embodiment, at least a portion of the second surface is formed with a reflective surface.

In an embodiment, the reflective surface is formed by applying a reflective laminate.

In an embodiment, applying the reflective laminate includes applying at least one of a gold-based and silver-based coating.

In an embodiment, the reflective laminate includes directing a flux of particles at the second surface with the assistance of a flux of ions.

In an embodiment, applying the reflective laminate includes applying reflective paint.

In an embodiment, the transmission input of the at least one collection waveguide and a transmission output of the at least one delivery waveguide are spaced apart at a predetermined distance to facilitate collection of radiation emitted from tissue of a predetermined scope and depth from the flexible, expandable first surface.

In an embodiment, at least one of the collection waveguides or delivery waveguides is a fiber optic manufactured to distribute or collect radiation about at least a 90 degree circumferential perimeter of its tip.

In an embodiment, at least one of the collection waveguides or delivery waveguides is a fiber optic manufactured by forming a recess out of its tip.

In an embodiment, the recess is formed by chemical etching.

In an embodiment, the fiber optic is a graded-index core optical fiber in which the chemical etching selectively removes dopant material to form the recess.

In an embodiment, at least one of the at least one delivery waveguide and at least one collection waveguide have a core diameter of 50 microns or less.

In an embodiment, the first surface and the second surface are arranged so as to hold the tip of the at least one delivery waveguide and the at least one collection waveguide at a predetermined distance from the first surface when the first surface is fully expanded.

In an embodiment, the first surface is attached to the second surface at discrete locations circumferentially distributed about the inner circumference of the first surface and wherein the second surface is attached to the flexible conduit at discrete locations circumferentially distributed about the circumference of the flexible conduit, wherein the discrete locations circumferentially distributed about the inner circumference of the first surface are circumferentially offset from the discrete locations circumferentially distributed about the inner circumference.

In an embodiment, the at least one waveguide is arranged to be slidably moveable along the flexible conduit.

In an embodiment, a mechanical locking mechanism is fixedly attached to the at least one waveguide so as to allow an operator to slidably manipulate the waveguide.

In an embodiment, the at least one waveguide that is slidably movable is placed in a sleeve, the sleeve coupled to the second surface and wherein the at least one waveguide is slidably movable within the sleeve.

In an embodiment, the sleeve is constructed of translucent material.

Other advantages and novel features, including optical methods and designs of illuminating and collecting an optical signal of a lumen wall through a lumen-expanding balloon, are described within the detailed description of the various embodiments of the present specification.

### BRIEF DESCRIPTION OF THE DRAWINGS

The foregoing and other objects, features, and advantages of the invention will be apparent from the more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A is an illustrative view of a catheter instrument for analyzing and medically treating a lumen, according to an embodiment of the present invention.

FIG. 1B is a block diagram illustrating an instrument deployed for analyzing and medically treating the lumen of a patient, according to an embodiment of the present invention.

FIG. 2A is an expanded illustrative view of the treatment end of a catheter instrument according to an embodiment of the present invention.

FIG. 2B is a cross-sectional view of the catheter of FIG. 2A, taken along section lines I-I′ of FIG. 2A.

FIG. 2C is a cross-sectional view of the catheter of FIG. 2A, taken along section lines II-II′ of FIG. 2A.

FIG. 2D is a cross-sectional view of the catheter of FIG. 2A, taken along section lines III-III′ of FIG. 2A.

FIGS. 3A-3F are cross-sectional views illustrating sequential steps of performing a balloon angioplasty procedure according to embodiments of the present invention.

FIG. 4A is an illustrative schematic view of a fiber tip being formed in an etchant solution in a method according to an embodiment of the invention.

FIG. 4B is an illustrative view of the fiber tip of FIG. 4A, while placed in an etchant solution according to an embodiment of the invention.

FIG. 4C is an illustrative schematic view of the fiber tip of FIG. 4A after extraction from an etchant solution.

FIG. 4D is an illustrative schematic view of a of a recessed fiber tip being placed in a sealant solution.

FIG. 4E is an illustrative schematic view of the fiber tip of FIG. 4D after extraction from the sealant solution of FIG. 4D.

FIG. 5A is an expanded illustrative view of the treatment end of a catheter instrument according to another embodiment of the present invention.

FIG. 5B is a cross-sectional view of the catheter of FIG. 5A, taken along section lines I-I′ of FIG. 5A.

FIG. 5C is a cross-sectional view of the catheter of FIG. 5A, taken along section lines II-II′ of FIG. 5A.

FIG. 5D is a cross-sectional view of the catheter of FIG. 5A, taken along section lines III-III′ of FIG. 5A.

FIG. 6A is an expanded illustrative view of the treatment end of a catheter instrument according to another embodiment of the present invention.

FIG. 6B is a cross-sectional view of the catheter of FIG. 6A, taken along section lines I-I′ of FIG. 6A.

FIG. 6C is a cross-sectional view of the catheter of FIG. 6A, taken along section lines II-II′ of FIG. 6A.

FIG. 6D is a cross-sectional view of the catheter of FIG. 6A, taken along section lines III-III′ of FIG. 6A.

FIG. 7A is an expanded illustrative view of the treatment end of a catheter instrument according to another embodiment of the present invention.

FIG. 7B is an expanded illustrative view of the treatment end of a catheter instrument according to another embodiment of the present invention.

FIG. 7C is a cross-sectional view of the catheter of FIGS. 7A and 7B, taken along section lines I-I′ of FIGS. 7A and 7B.

FIG. 7D is a cross-sectional view of the catheter of FIG. 7B, taken along section lines II-II′ of FIG. 7B.

FIG. 7E is a cross-sectional view of the catheter of FIG. 7A, taken along section lines III-III′ of FIG. 7A.

FIG. 8A is an expanded illustrative view of the treatment end of a catheter instrument according to another embodiment of the present invention.

FIG. 8B is a cross-sectional view of the catheter of FIG. 8A, taken along section lines I-I′ of FIG. 8A.

FIG. 8C is a cross-sectional view of the catheter of FIG. 8A, taken along section lines II-II′ of FIG. 8A.

FIG. 8D is an expanded illustrative view of a catheter in accordance with FIG. 8A and a means for attaching catheter components according to an embodiment of the invention.

FIG. 9A is an expanded illustrative view of the treatment end of a catheter instrument according to another embodiment of the present invention.

FIG. 9B is a cross-sectional view of the catheter of FIG. 9A, taken along section lines I-I′ of FIG. 9A.

FIG. 9C is a cross-sectional view of the catheter of FIG. 9A, taken along section lines II-II′ of FIG. 9A.

FIG. 9D is a cross-sectional view of the catheter of FIG. 9A, taken along section lines II-II′ of FIG. 9A.

FIG. 10A is an expanded illustrative view of the treatment end of a catheter instrument according to another embodiment of the present invention.

FIG. 10B is a cross-sectional view of the catheter of FIG. 10A, taken along section lines I-I′ of FIG. 10A.

FIG. 10C is a cross-sectional view of the catheter of FIG. 10A, taken along section lines II-II′ of FIG. 10A.

FIG. 11A is an expanded illustrative view of the treatment end of a catheter instrument according to another embodiment of the present invention.

FIG. 11B is a cross-sectional view of the catheter of FIG. 11A, taken along section lines I-I′ of FIG. 11A.

FIG. 11C is a cross-sectional view of the catheter of FIG. 11A, taken along section lines II-II′ of FIG. 11A.

FIG. 11D is a cross-sectional view of the catheter of FIG. 11A, taken along section lines III-III′ of FIG. 11A.

FIG. 12A is an expanded illustrative view of the treatment end of a catheter instrument according to another embodiment of the present invention.

FIG. 12B is a cross-sectional view of the catheter of FIG. 12A, taken along section lines I-I′ of FIG. 12A.

FIG. 12C is a cross-sectional view of the catheter of FIG. 12A, taken along section lines II-II′ of FIG. 12A.

FIG. 12D is a cross-sectional view of the catheter of FIG. 12A, taken along section lines III-III′ of FIG. 12A.

FIG. 13A is an expanded illustrative view of the treatment end of a catheter instrument according to another embodiment of the present invention.

FIG. 13B is a cross-sectional view of the catheter of FIG. 13A, taken along section lines I-I′ of FIG. 13A.

FIG. 13C is a cross-sectional view of the catheter of FIG. 13A, taken along section lines II-II′ of FIG. 13A.

FIG. 14A is an expanded illustrative view of the treatment end of a catheter instrument according to another embodiment of the present invention.

FIG. 14B is a cross-sectional view of the catheter of FIG. 14A, taken along section lines I-I′ of FIG. 14A.

FIG. 14C is a cross-sectional view of the catheter of FIG. 14A, taken along section lines II-II′ of FIG. 14A.

FIG. 15A is an expanded illustrative view of the treatment end of a catheter instrument according to another embodiment of the invention.

FIG. 15B is a cross-sectional view of the catheter of FIG. 15A, taken along section lines I-I′ of FIG. 15A.

FIG. 15C is another embodiment of a cross-sectional view of the catheter of FIG. 15A, taken along section lines I-I′ of FIG. 15A.

FIG. 16A is an expanded illustrative view of the treatment end of a catheter instrument according to another embodiment of the present invention.

FIG. 16B is a cross-sectional view of the catheter of FIG. 16A, taken along section lines I-I′ of FIG. 16A.

FIG. 16C is a cross-sectional view of the catheter of FIG. 16A, taken along section lines II-II′ of FIG. 16A.

FIG. 16D is a cross-sectional view of the catheter of FIG. 16A, taken along section lines III-III′ of FIG. 16A.

FIG. 16E is another expanded illustrative view of the treatment end of the catheter of FIG. 16.

FIG. 16F is an expanded illustrative cutout view of the catheter of FIG. 16A.

FIGS. 16G and 16H are illustrative cross-sectional views of the catheter instrument of FIG. 16A within a lumen.

FIG. 16I is a chart of absorption measurements comparing radiation at various wavelengths traveling through water across a 1 mm span.

FIG. 16J is an illustrative schematic of an optical source and detector configuration during another step of the operation of the catheter of FIG. 16A according to an embodiment of the invention.

FIG. 16K is an illustrative schematic of an optical source and detector configuration during another step of the operation of the catheter of FIG. 16A according to an embodiment of the invention.

FIG. 16L is an illustrative schematic of an optical source and detector configuration during another step of the operation of the catheter of FIG. 16A according to an embodiment of the invention.

FIG. 16M is an illustrative schematic of an optical source and detector configuration during another step of the operation of the catheter of FIG. 16A according to an embodiment of the invention.

FIG. 17A is an illustrative schematic of another embodiment of a catheter configuration including two delivery fibers and two collection fibers contiguous with the guidewire sheath for detecting balloon underexpansion.

FIG. 17B is an illustrative cross-sectional schematic of the delivery fibers and collection fibers positioned for analyzing the expansion profile of the balloons of FIG. 17A within a lumen.

FIG. 18A is an illustrative schematic of another embodiment of a catheter configuration including two delivery fibers and two collection fibers positioned along a surface of the balloon.

FIG. 18B is an illustrative cross-sectional schematic of the delivery fibers and collection fibers positioned for analyzing the expansion profile of the balloons of FIG. 18A within a lumen.

FIGS. 19A and 19D are illustrative views of the treatment end of a catheter instrument with slidably movable fibers according to an embodiment of the present invention.

FIG. 19B is an illustrative view of the treatment end of the catheter instrument of FIG. 19A with fibers moved to approximately the longitudinal center of balloon.

FIG. 19C is an illustrative view of the treatment end of the catheter instrument of FIG. 19A with fibers positioned near the proximal end of balloon.

FIG. 19E is a cross-sectional view of the catheter of FIG. 19D, taken along section lines I-I′ and II-II′ of FIG. 19D.

FIG. 20A is an illustrative view of the treatment end of a catheter instrument with slidably movable fibers according to another embodiment of the present invention.

FIG. 20B is a cross-sectional view of the catheter of FIG. 20A, taken along section lines I-I′ of FIG. 20A.

FIG. 21A is another illustrative view of an arrangement of slidably movable fibers integrated with an inflatable balloon catheter.

FIG. 21B is another illustrative view of an arrangement of slidably movable fibers integrated with an inflatable balloon.

FIG. 21C is an illustrative view of a section of a catheter 430 having guidewire lumen opening according to an embodiment of the invention.

FIG. 22A is an illustrative view of the proximate end of a catheter instrument for manipulating slidable fibers according to an embodiment of the invention.

FIG. 22B is a cross-sectional illustrative view of the catheter instrument of FIG. 22A.

FIG. 22C is an illustrative cross-sectional view of the catheter instrument of FIGS. 22A-B across section lines I-I′ of FIG. 22B.

FIG. 22D is an illustrative view of proximate end the catheter instrument with a fiber-sliding section in an extended position.

### DETAILED DESCRIPTION OF EMBODIMENTS OF THE INVENTION

The accompanying drawings are described below, in which example embodiments in accordance with the present invention are shown. Specific structural and functional details disclosed herein are merely representative. This invention may be embodied in many alternate forms and should not be construed as limited to example embodiments set forth herein. Accordingly, specific embodiments are shown by way of example in the drawings. It should be understood, however, that there is no intent to limit the invention to the particular forms disclosed, but on the contrary, the invention is to cover all modifications, equivalents, and alternatives falling within the spirit and scope of the claims. Like numbers refer to like elements throughout the description of the figures.

It will be understood that, although the terms first, second, etc. may be used herein to describe various elements, these elements should not be limited by these terms. These terms are used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element is referred to as being “on,” “connected to” or “coupled to” another element, it can be directly on, connected to or coupled to the other element or intervening elements may be present. In contrast, when an element is referred to as being “directly on,” “directly connected to” or “directly coupled to” another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments and is not intended to be limiting of the invention. As used herein, the singular forms “a,” “an” and “the” are intended to include the plural forms as well, unless the context clearly indicates otherwise. It will be further understood that the terms “comprise,” “comprises,” “comprising,” “include,” “includes” and/or “including,” when used herein, specify the presence of stated features, integers, steps, operations, elements, and/or components, but do not preclude the presence or addition of one or more other features, integers, steps, operations, elements, components, and/or groups thereof.

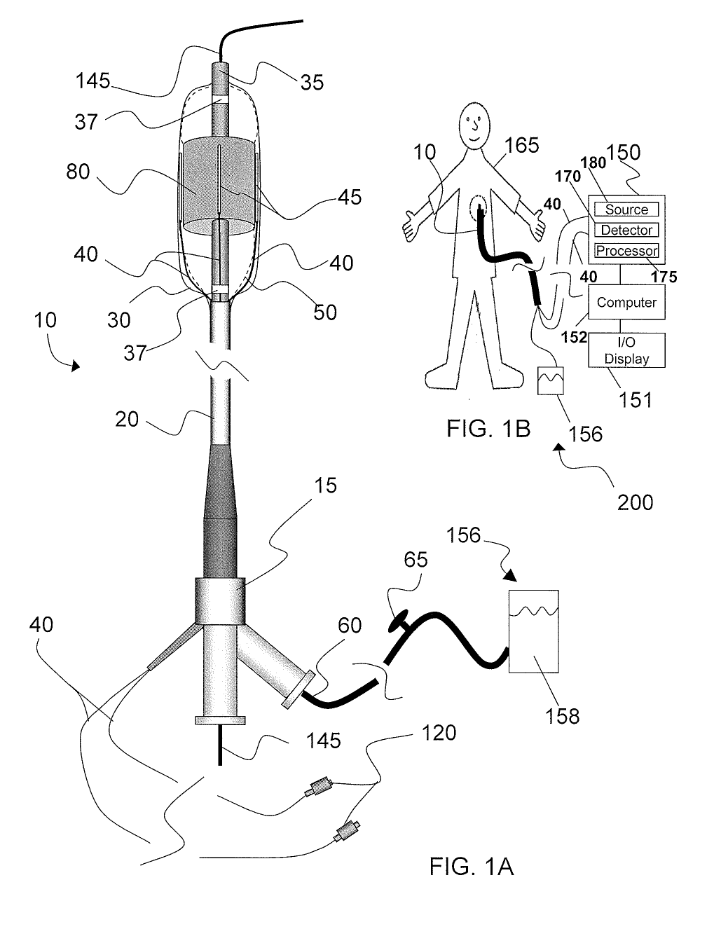

FIG. 1A is an illustrative view of a catheter instrument 10 for analyzing and medically treating a lumen, according to an embodiment of the present invention. FIG. 1B is a block diagram illustrating an instrument 200 deployed for analyzing and medically treating the lumen of a patient, according to an embodiment of the present invention. The catheter assembly 10 includes a catheter sheath 20 with at least two fibers 40, including one or more delivery fiber(s) connected to at least one source 180 and one or more collection fiber(s) connected to at least one detector 170. Catheter sheath 20 includes a guidewire sheath 35 and guidewire 145. The distal end of catheter assembly 10 includes an inner balloon 50 and a flexible outer covering 30. In an embodiment, inner balloon 50 and outer covering 30 function as a lumen expanding balloon (e.g., an angioplasty balloon).

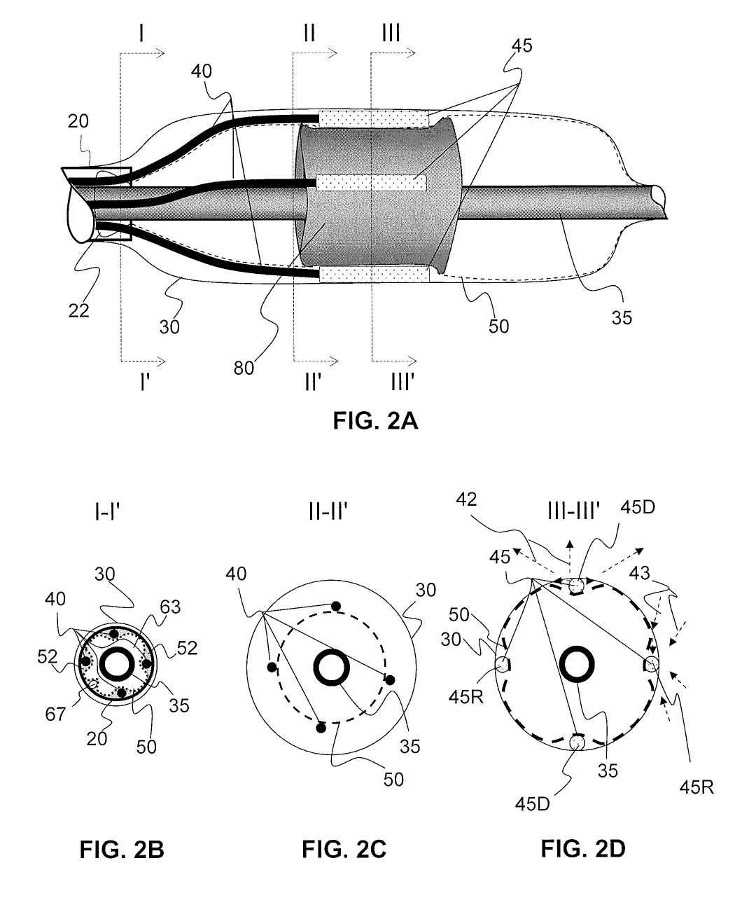

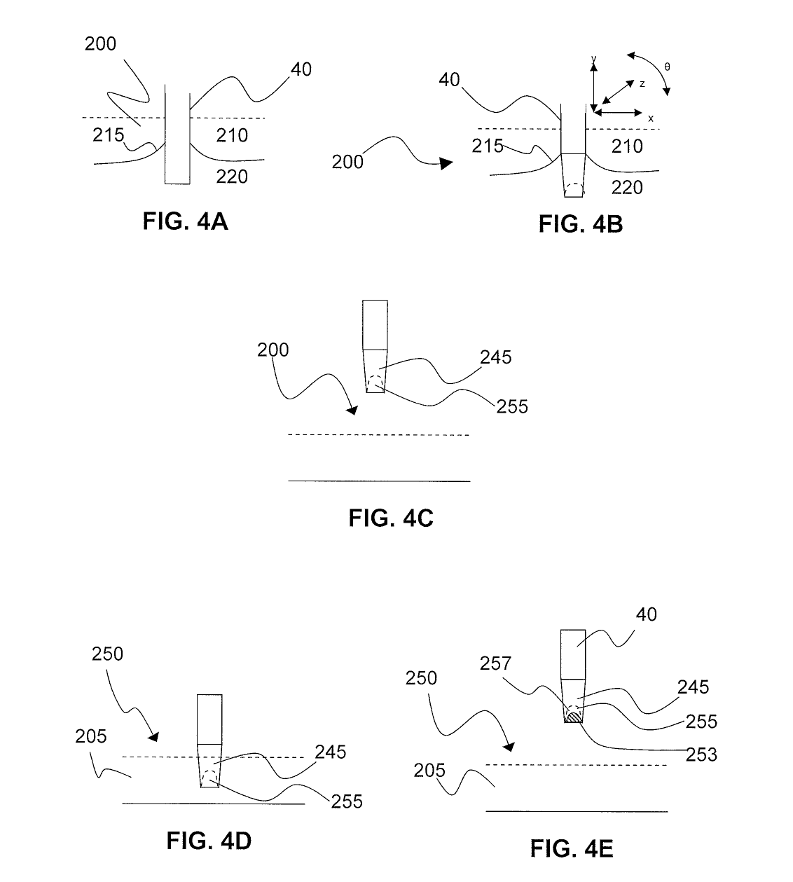

Delivery and collection ends 45 of fibers 40 are positioned between the inner balloon 50 and outer covering 30. Inner balloon 50 can include a reflective surface 80 so as to improve light delivery and collection to and from delivery/collection ends 45. The reflective surface 80 can be applied, for example, as a thin coating of reflective material such as, for example, gold-based or silver-based paint or laminate or other similar material. Outer covering 30 is comprised of a material translucent to radiation delivered and collected by fibers 40 such as, for example, translucent nylon or other polymers. The delivery and collection ends 45 are preferably configured to deliver and collect light about a wide angle such as, for example, between about at least a 120 to 180 degree cone around the circumference of each fiber, directed radially outward from about the center of catheter 10 such as exemplified in FIG. 2D and as further described herein below. Various methods for forming such delivery and collection ends are described in more detail herein (e.g., see FIGS. 4A-4E and accompanying description herein). Various such embodiments in accordance with the invention allow for diffusely reflected light to be readily delivered and collected between fibers 40 via tissue surrounding the catheter 10.

The proximate end of balloon catheter assembly 10 includes a junction 15 that distributes various conduits between catheter sheath 20 to external system components. Fibers 40 can be fitted with connectors 120 (e.g. FC/PC type) compatible for use with light sources, detectors, and/or analyzing devices such as spectrometers. Two radiopaque marker bands 37 are fixed about guidewire sheath 35 in order to help an operator to obtain information about the location of catheter 10 in the body of a patient (e.g. with the aid of a fluoroscope).

The proximate ends of fibers 40 are connected to a light source 180 and/or a detector 170 (which are shown integrated with an analyzer/processor 150). Analyzer/processor 150 can be, for example, a spectrometer which includes a processor 175 for processing/analyzing data received through fibers 40. A computer 152 connected to analyzer/processor 150 can provide an interface for operating the instrument 200 and to further process spectroscopic data (including, for example, through chemometric analysis) in order to diagnose and/or treat the condition of a subject 165. Input/output components (I/O) and viewing components 151 are provided in order to communicate information between, for example, storage and/or network devices and the like and to allow operators to view information related to the operation of the instrument 200.

Various embodiments provide a spectrometer (e.g., as analyzer/processor 150) configured to perform spectroscopic analysis within a wavelength range between about 250 and 2500 nanometers and include embodiments having ranges particularly in the near-infrared spectrum between about 750 and 2500 nanometers. Further embodiments are configured for performing spectroscopy within one or more subranges that include, for example, about 250-930 nm, about 1100-1385 nm, about 1600-1850 nm, and about 2100-2500 nm. Various embodiments are further described in, for example, previously cited and co-pending U.S. application Ser. No. 11/537,258 (entitled “SYSTEMS AND METHODS FOR ANALYSIS AND TREATMENT OF A BODY LUMEN”), and U.S. application Ser. No. 11/834,096 (entitled “MULTI-FACETED OPTICAL REFLECTOR”), the entire contents of each of which is herein incorporated by reference.

Junction 15 includes a flushing port 60 for supplying or removing fluid media (e.g., liquid/gas) 158 that can be used to expand or contract inner balloon 50 and, in an embodiment, an outer balloon formed by flexible outer covering 30. Fluid media 158 is held in a tank 156 from which it is pumped in or removed from the balloon(s) by actuation of a knob 65. Fluid media 158 can alternatively be pumped with the use of automated components (e.g. switches/compressors/vacuums). Solutions for expansion of the balloon are preferably non-toxic to humans (e.g. saline solution) and are substantially translucent to the selected light radiation.

FIG. 2A is an expanded illustrative view of the treatment end of a catheter instrument 10 according to an embodiment of the present invention. FIG. 2B is a cross-sectional view of the catheter of FIG. 2A, taken along section lines I-I′ of FIG. 2A. FIG. 2C is a cross-sectional view of the catheter of FIG. 2A, taken along section lines II-II′ of FIG. 2A. FIG. 2D is a cross-sectional view of the catheter of FIG. 2A, taken along section lines III-III′ of FIG. 2A. In an embodiment, a flexible outer covering 30 can operate as an inflatable balloon and is attached at its proximate end about the distal end of catheter sheath 20. Inner balloon 50 and fibers 40 extend through an opening 22 at the distal end of catheter sheath 20 and into balloon 50. In an embodiment, the proximate end of inner balloon 50 is attached to the inside of catheter sheath 20 with adhesive 52 placed between inner balloon 50 and catheter sheath 20. An intervening lumen between catheter sheath 20 and guidewire sheath 35 can be used to transfer fluid media through an opening 63 between inner balloon 50 and a fluid source (e.g., liquid/gas source 156 of FIGS. 1A-1B). A separate lumen 67 can be used to transfer fluid to and from the area between outer covering 30 (e.g., as in an angioplasty balloon) and inner balloon 50.

In an embodiment, both inner balloon 50 and lumen 67 are supplied simultaneously by the same fluid source (e.g., liquid/gas source 156). Inner balloon 50 is initially filled with fluid and will continue to expand against outer covering 30 as fluid pressure between inner balloon 50 and guidewire sheath 35 and the fluid pressure between the outer covering 30 and inner balloon 50 equalize, resulting in the distal end acting as an angioplasty balloon while substantially maintaining the delivery and collection ends 45 of fibers 40 against the inside wall of outer covering 30.

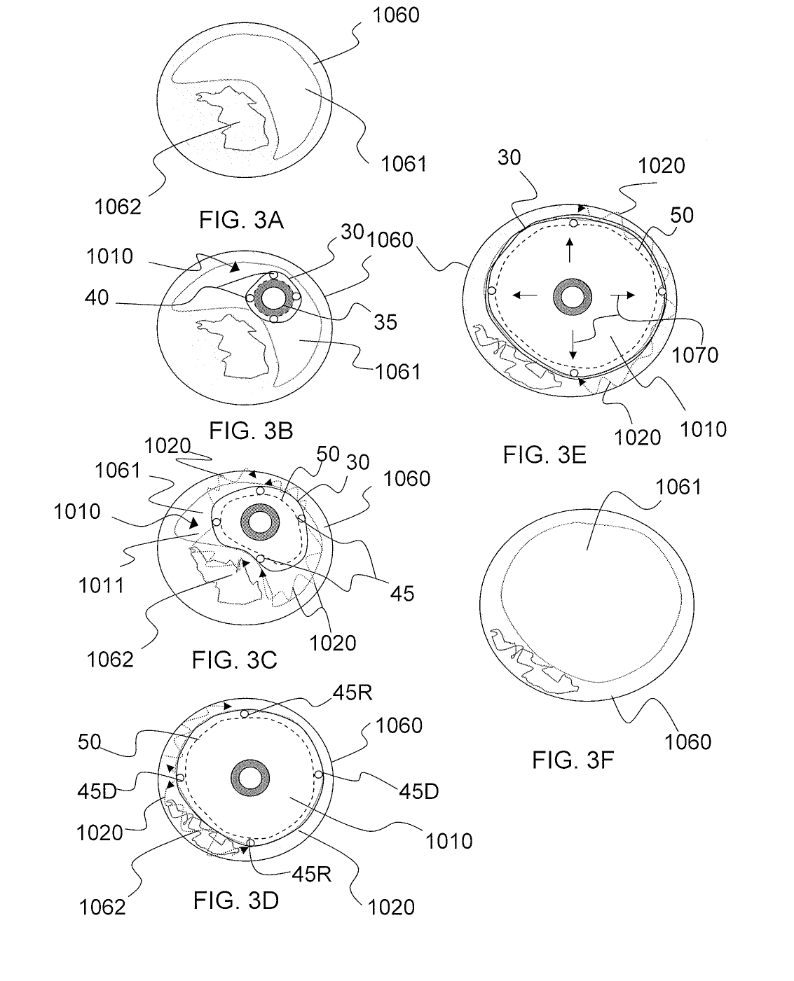

FIGS. 3A-3F are cross-sectional views illustrating the sequential steps of performing a balloon angioplasty procedure, in accordance with an embodiment of the present invention. FIG. 3A is a cross-sectional view of a constricted body lumen 1061 having a lumen wall 1060. The lumen 1061 may be constricted due to a blockage, for example a blockage 1062 caused by an accumulation of lipid content.

As shown in FIG. 3B, a balloon catheter 1010, for example of various embodiments described herein, is inserted into the constricted lumen 1061 in accordance with conventional procedures. In one embodiment, the balloon catheter 1010 comprises a core guidewire lumen 35, an outer covering 30, an inner balloon 50 (shown by dotted line), and at least one delivery/collection fiber 40. During a treatment procedure, the physician first inserts a guidewire into the constricted lumen 1061 via a puncture point such as, for example, located at the groin or wrist. Next, the physician places the balloon catheter 1010 on the guide wire. The balloon catheter 1010 comprises a balloon mechanism with an outer covering 30 that, upon entry to the constricted lumen 1061, is in an unexpanded state.

As shown in FIG. 3C, the positioned balloon catheter 1010 is partially inflated by delivering fluid through a port into the balloon catheter 1010 (as further described in reference to various embodiments herein). The catheter 1010 enables the collection of data of the spectral features of the lumen wall 1060 by delivering optical radiation 1020 from a delivery fiber to the lumen wall, and collecting optical radiation 1020 that is emitted from the lumen wall and received by a collection fiber. The collection of data of the spectral features of the lumen wall 1060 are used to determine the position of the balloon catheter 1010 with respect to a target region. Since the lumen wall information is obtained via spectral analysis in real-time, the physician can rely on this information to determine the relative position and type of diseased area 1062 of the lumen, and, accordingly, help determine the necessary procedure (e.g. balloon angioplasty, stent insertion (including the type of stent), bypass, and/or systemic drug therapy). The operator could decide, for example, to cease inflation and withdraw the catheter from the patient based on signals provided from the radiation 1020 that are, for example, indicative of a lesion highly prone to rupture.

In addition, signals 1020 from catheter 1010 can be used to more properly control the inflation of catheter 1010. An operator can gradually inflate balloon catheter 1010 while the system monitors signals 1020 for the presence of blood and proximity of the vessel wall to the balloon wall. In addition, signals can be measured for the presence of inflation media. If a relatively significant level of blood is detected about the entire periphery of catheter 1010 and outer covering 30, the balloon catheter is not likely sufficiently expanded for the applicable purpose (e.g., angioplasty, pre-stenting dilation, stent deployment, and/or post-stenting expansion). When the signal for blood has substantially diminished, the operator can further controllably inflate catheter 1010 to an appropriate level.

In an embodiment, spectroscopy is employed with one or more wavelengths with predetermined spectra profiles known to have at least a nominally predictable relationships with the content of adjacent blood alone or tissue and/or balloon inflation media. In an embodiment, one or more wavelengths selected from 407, 532, and between about 800 and 1000 nanometers are spectroscopically analyzed. In an embodiment, diffuse reflectance spectroscopy is used. In an embodiment, ratios between two or more of these wavelengths are previously measured at various blood depths apart from a vessel wall, programmed into a system, and later compared to in-process data collected during an actual procedure. In an embodiment, the one or more wavelengths consist of wavelengths of 532 and 407 nanometers and in another embodiment consist of 532 and 800 nanometers.

Normally, typical angioplasty-type procedures rely on inaccurate fluoroscopy measurements and balloon expansion profiles made prior to catheter deployment to determine the level of fluid pressure/inflation needed. In order to avoid risky complications, these traditional procedures often overinflate the balloon catheter. An underexpanded stent, for example, may not only fail to properly support a targeted vessel area but also cause additional undesired blockages itself. Overexpansion, however, presents its own risks (e.g. rupture and other vessel damage) and an angioplasty-type procedure may therefore be avoided altogether as a treatment. Various embodiments of the invention as described herein can help avoid these occurrences by more accurately determining apposition of the catheter balloon against a vessel wall in real-time.

A signal 1020 indicative of the presence of blood about only portions of catheter 1010 could also be used to help determine, for example, the presence and peripheral location of a hard (e.g., calcified) lesion. If the localized presence of blood is detected when the balloon should be substantially apposed to lumen wall 1060, the signals may be indicative of a deformed mal-apposed balloon that may result when such hard lesions significantly resist expansion while other portions of the vessel do not so resist. Under these circumstances, the mal-apposed balloon may either trap blood in pockets between the balloon wall and the vessel wall or allow blood to freely flow by along certain portions of the balloon. Signals 1020 could further verify the presence of, for example, such elements as calcium or other elements indicative of hard lesions. Since an embodiment of the invention can also identify weaknesses along the lumen wall prior to fully deploying an angioplasty balloon at a target region of the lumen wall, the embodiment can reduce the risk of a rupture occurring at or near the blockage 1062 during or after an angioplasty procedure.

As shown in FIG. 3D, the catheter 1010 is shown further inflated and substantially apposed to lumen 1061 at the target region for treatment (e.g., balloon angioplasty and/or stent insertion (stent not shown)). Optical radiation 1020 is transmitted from a distal end of the delivery fiber and transmitted through the balloon catheter 1010 to the catheter surface that abuts the lumen wall 1060. The optical radiation passes through the surface of outer covering 30 and impinges the target region of the lumen wall 1060 and can interact with the tissue/fluids therein in the manner of, for example, fluorescence, luminescence, and/or diffuse reflectance as described in detail herein. Collection fibers can receive the emitted optical radiation from the lumen wall 1060 and transfer them to one or more detectors and for further processing (e.g., a spectroscopic analysis system). In order to separately process and assess signals from a particular circumferential portion of lumen 1060, an embodiment activates, e.g., supplies light to, delivery fiber(s) 45 while other delivery fiber(s) 45D are deactivated by the system. Since the balloon catheter 1020 is in direct contact with the lumen wall, such that little or no blood is between the balloon and the lumen wall, high-quality spectral data can be obtained. This additional spectral data allows the physician to receive in real-time the treatment results, as well as current physiological and pathological changes on the treatment.

For example, if a lumen is being inspected in an angioplasty application (e.g., pre-dilation, stenting, post-dilation), the physician can rapidly make a decision for subsequent therapy, e.g., a stent insertion and/or a drug local injection therapy after a sample balloon angioplasty for second treatment. The spectral data can also indicate the preferred stent to be selected for treatment, of any required future treatment, etc. by analyzing pathology results on the lumen wall. The spectral data can also be stored for future analysis or comparison to current treatment(s). In an embodiment, at the point when catheter 1020 substantially apposes the lumen wall (e.g., as shown in FIG. 3D), the physician can use the balloon's expansion profile and collected data to determine whether and how much further to inflate the balloon catheter for an applicable treatment.

In an embodiment, selected drugs (not shown) are coated over the outside covering 30 of balloon catheter 1010. In an embodiment, one or more of the drugs coating covering 30 can be activated, e.g., so as to provide therapeutic effect, by the emission of selected radiation from fiber ends 45 to the covering 30 at various stages of the deployment of catheter 1010. A physician, for example, can use information gathered from prior analysis performed by a balloon catheter 1010 to decide whether and if selected drugs should be activated or left inactivated.

As shown in FIG. 3E, balloon catheter 1010 is further inflated and dilating lumen 1060 as in, for example, an angioplasty. Further data can be collected through the fiber optical system in order to monitor and assess the ongoing treatment. The treated and analyzed lumen 1060 is shown in FIG. 3F after deflation and removal of balloon catheter 1010.

FIG. 4A is an illustrative schematic view of a fiber tip being formed in an etchant solution in a method according to an embodiment of the invention. FIG. 4B is an illustrative view of the fiber tip of FIG. 4A, while placed in an etchant solution according to an embodiment of the invention. FIG. 4C is an illustrative schematic view of the fiber tip of FIG. 4A after extraction from an etchant solution. FIG. 4D is an illustrative schematic view of a recessed fiber tip being placed in a sealant solution. FIG. 4E is an illustrative schematic view of the fiber tip of FIG. 4D after extraction from the sealant solution of FIG. 4D.

In an embodiment, the process for forming a fiber tip 345 occurs (as shown in FIG. 4A) by placing the end of a fiber 40 in a bath 200 including an etchant 220. An organic solvent 210 (e.g., silicone) can be included in the bath so as to control formation of a meniscus 215 and to prevent inadvertent exposure of portions of fiber 40 to the etchant. Depending on the fiber type and the desired profile/shape of tip 245, fiber 40 is held in bath 200 of etchant solution for a predetermined amount of time. In an embodiment, fiber 40 has a graded index core with a diameter of between about 50 and 100 microns and is held in an etchant comprising HF for a period between about 4 minutes to 15 minutes or more. Fiber 40 can also be moved and repositioned in the etchant to effect the shape of tip 245 such as illustrated in FIG. 4B. As illustrated in FIG. 4C, etchant solution 220 gradually removes material from the cladding/core interior of fiber tip 245, forming a shaped recess 255 within the cladding/core interior. Methods for shaping fiber tips in this manner are more fully described in U.S. Application No. 61/025,514, entitled “BODY LUMEN PROBES WITH SHAPED FIBER ENDS”, filed Feb. 1, 2008, and U.S. Patent Application No. 61/082,721 filed Jul. 22, 2008, the entire contents of each of which is herein incorporated by reference.

Referring in particular to FIGS. 4D and 4E, a fiber tip 245 with a shaped recess such as, for example, recess 255 shown in FIG. 4C is placed in a sealant bath 250 of sealant 205 so as to form a protective seal 253 across the opening of the recess and help prevent contaminants including, for example, fluid media from interfering with the optical functions of the fiber tip 245. In various embodiments, sealants for use in protecting recess 255 include, for example, pyroxylin, thermoplastics such as ethylene-vinyl acetate, and thermosetting plastics such as ultraviolet cured glass glue (e.g., Loctite brand series 3345, e.g., see http://www.loctite.com). Referring in particular to FIG. 4E, after tip 245 is extracted from sealant bath 250, protective seal 253 is formed within recess 255. In an embodiment, an air gap 257 may be formed between the protective seal 253 and the surface of recess 255. Air gap 257 can, for example, aid in directing refracted light incident upon recess 255 toward directions oblique to the longitudinal axis of fiber tip 245.

Various other delivery and collection end arrangements of fibers 40 can be adapted for use in embodiments of the present invention such as, for example, those arrangements described in co-pending and related U.S. patent application Ser. No. 11/537,258, filed on Sep. 29, 2006, published as Patent Application Publication No. 2007/0078500 A1, the entire contents of which is incorporated herein by reference.

In embodiments, the recess 255 can have other shapes, such that a vertex is located within the core of the tip. In other embodiments, recess 255 can have other shapes that comprise higher order polynomial curves. In other embodiments, the recess has a curved surface, the curved surface having a vertex within the core.

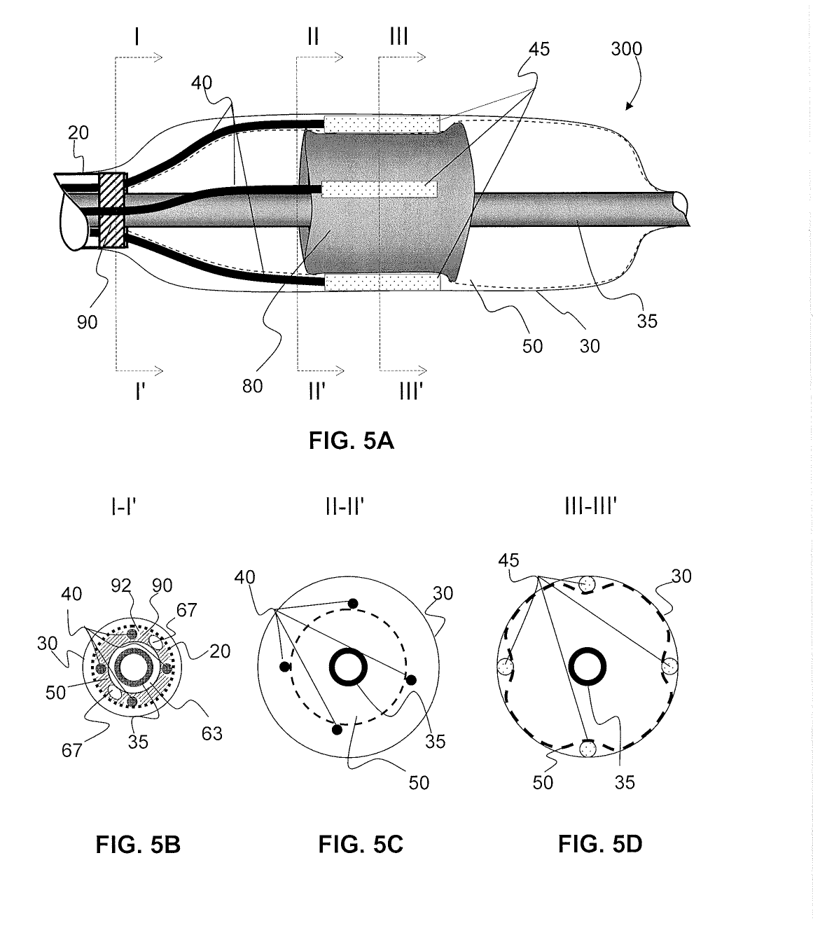

FIG. 5A is an expanded illustrative view of the treatment end of a catheter instrument 300 according to another embodiment of the present invention. FIG. 5B is a cross-sectional view of the catheter of FIG. 5A, taken along section lines I-I′ of FIG. 5A. FIG. 5C is a cross-sectional view of the catheter of FIG. 5A, taken along section lines II-II′ of FIG. 5A. FIG. 5D is a cross-sectional view of the catheter of FIG. 5A, taken along section lines III-III′ of FIG. 5A. As an alternative to using adhesive 52, for example, as shown in FIG. 2B, a ring element 90 holds fibers 40 in grooves 92 abutted by catheter body 20. Holes 67 provide for the transfer of inflation media (not shown) to and from the space between inner balloon 50 and outer covering 30. An intervening opening 63 between the inner wall of ring 90 and guidewire sheath 35 provides a conduit through which inflation media is transferred to and from inner balloon 50. Ring 90 can be molded as an integral part of catheter sheath 20, or can be separately assembled and affixed.

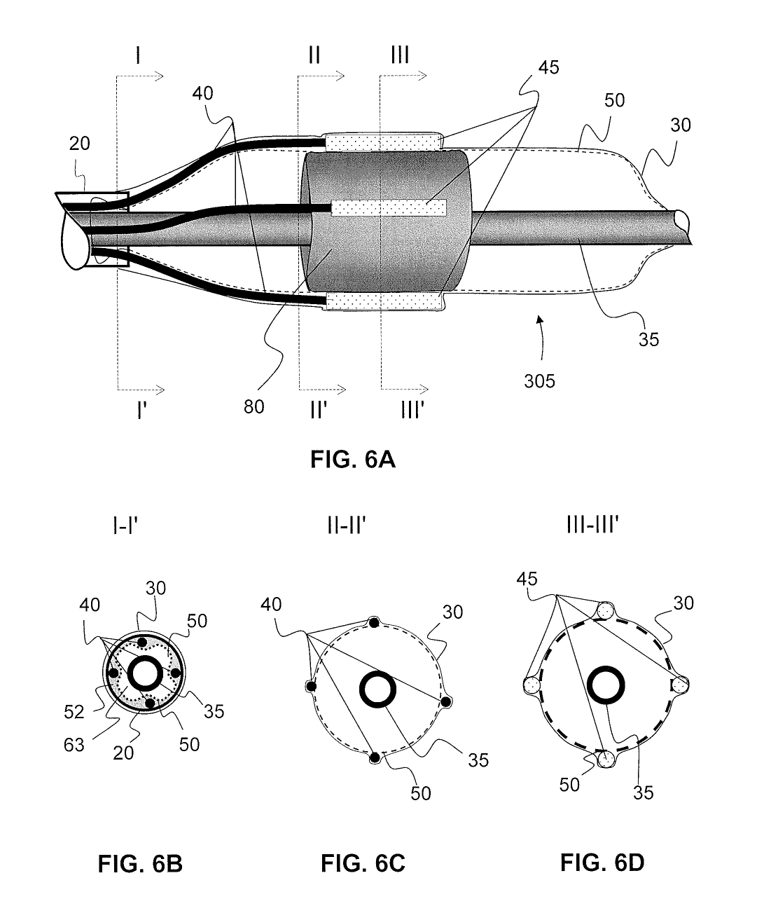

FIG. 6A is an expanded illustrative view of the treatment end of a catheter instrument 305 according to another embodiment of the present invention. FIG. 6B is a cross-sectional view of the catheter of FIG. 6A, taken along section lines I-I′ of FIG. 6A. FIG. 6C is a cross-sectional view of the catheter of FIG. 6A, taken along section lines II-II′ of FIG. 6A. FIG. 6D is a cross-sectional view of the catheter of FIG. 6A, taken along section lines III-III′ of FIG. 6A. As an alternative to inflating the intervening space between inner balloon 50 and outer covering 30 with fluid media, outer covering 30 generally acts only to protect fibers 40 from contact with external tissue and fluid and expands via pressure from inner balloon 50. Although the embodiment can necessitate fewer conduits (e.g., a lack of an additional lumen such as the lumen 67 of FIGS. 2B and 5B) and less complication for purposes of balloon inflation and fluid dynamics, e.g., such as directing the predominance of fluid flow to an inner balloon 50, additional pressure will be exerted upon fiber ends 45 between inner balloon 50 and the inner wall of the targeted body lumen, potentially increasing the likelihood of damage occurring to fiber ends 45.

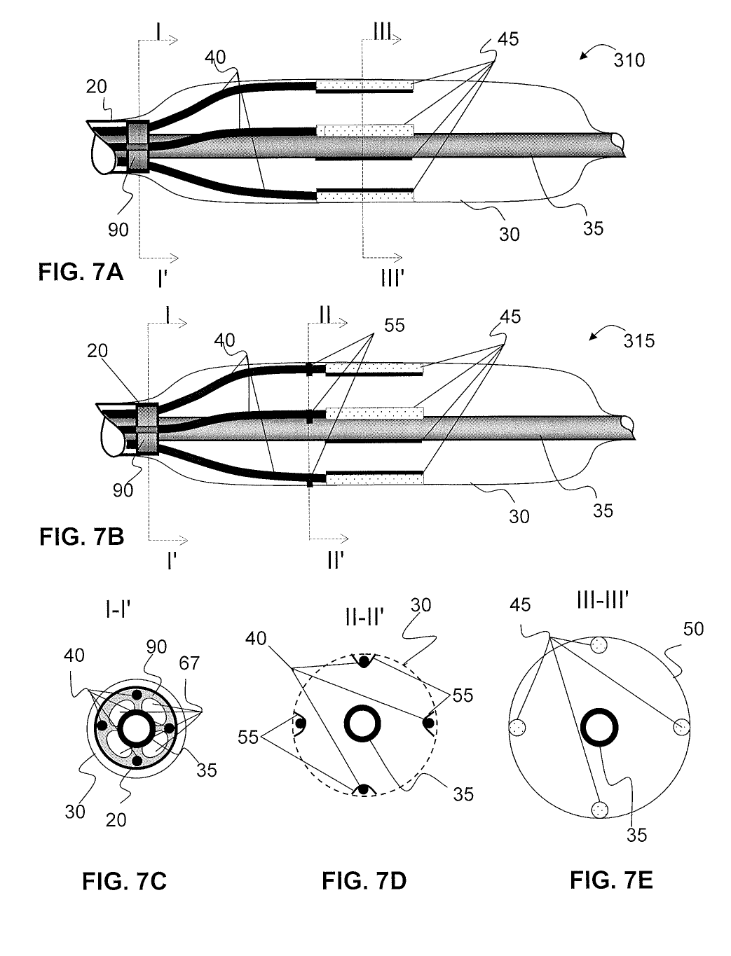

FIG. 7A is an expanded illustrative view of the treatment end of a catheter instrument 310 according to another embodiment of the present invention. FIG. 7C is a cross-sectional view of the catheter of FIG. 7A, taken along section lines I-I′ of FIG. 7A. FIG. 7E is a cross-sectional view of the catheter of FIG. 7A, taken along section lines III-III′ of FIG. 7A. The treatment end comprises a single balloon formed by outer covering 30 having an interior of which is affixed fiber ends 45. In an embodiment, a glue or epoxy or similar compound is used as an adhesive to affix fiber ends 45 to the balloon 30. The compound is preferably medical grade and highly translucent to radiation selected for delivery from or collection by fibers 40, of which numerous types are commercially available. The compound is also preferably highly flexible so as to forgive stresses caused by the expansion of balloon 30 during deployment. A ring element 90 is placed at the end of catheter sheath 20 through which fibers 40 pass and are generally distributed evenly about the inside circumference of balloon 30. Channels 67 provide a conduit through which fluid media is transferred to and from balloon 30.

FIG. 7B is an expanded illustrative view of the treatment end of a catheter instrument 315 according to another embodiment of the present invention. FIG. 7C is a cross-sectional view of the catheter of FIG. 7B in addition to the catheter of 7A, taken along section lines I-I′ of FIGS. 7A and 7B. FIG. 7D is a cross-sectional view of the catheter of FIG. 7B, taken along section lines II-II′ of FIG. 7A. As an alternative to directly adhering fibers 40 to balloon 30 by the embodiment as described in reference to FIG. 7A, semi-ring shaped fiber holders 55 are affixed to, for example, with a medical grade epoxy, or otherwise formed on the inside of balloon 30, and through which fibers 40 can movably slide, thus reducing stress placed on fibers 40 during the expansion of balloon 30.

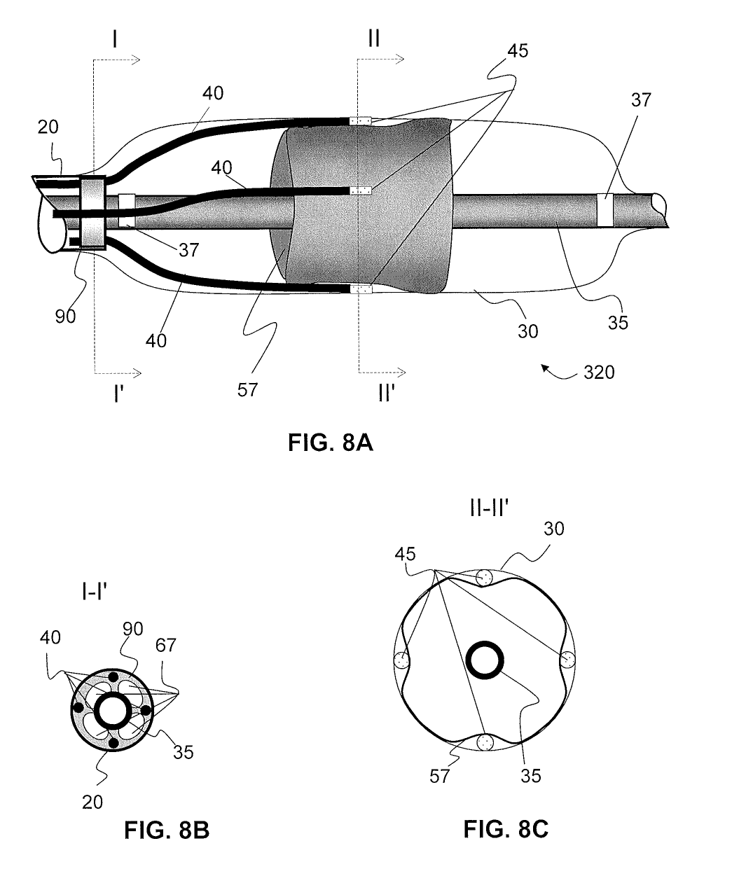

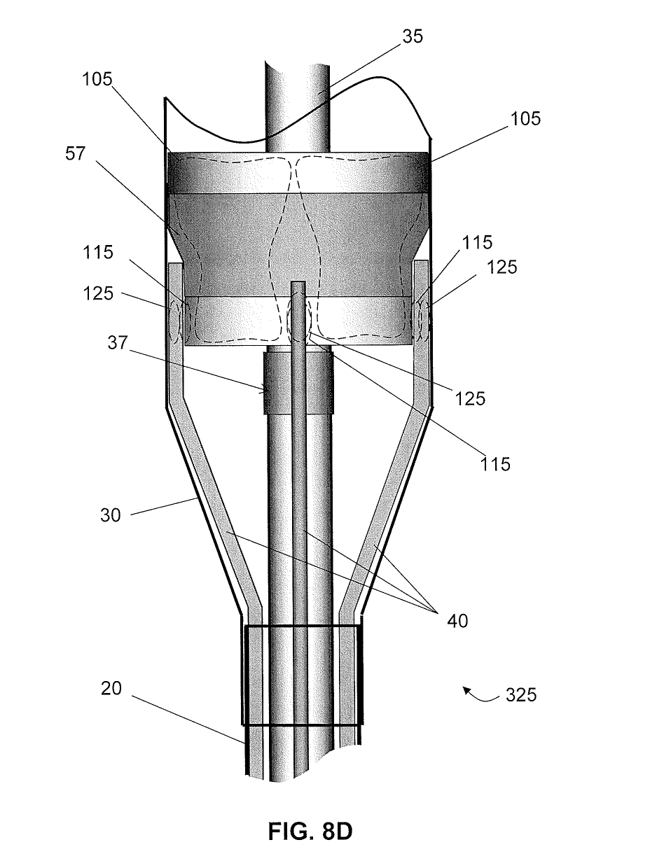

FIG. 8A is an expanded illustrative view of the treatment end of a catheter instrument 320 according to another embodiment of the present invention. FIG. 8B is a cross-sectional view of the catheter of FIG. 8A, taken along section lines I-I′ of FIG. 8A. FIG. 8C is a cross-sectional view of the catheter of FIG. 8A, taken along section lines II-II′ of FIG. 8A. FIG. 8D is an expanded illustrative view of a catheter in accordance with FIG. 8A and means for attaching catheter components according to an embodiment of the invention. A flexible reflective inner sheath 57 is positioned between fiber ends 45 and balloon 30. The outside surface of reflective sheath 57 provides a surface for reflecting and increasing light delivered and collected by fiber ends 45. In an embodiment, fiber sheath 57 is affixed to fiber ends 45 (which are also attached to balloon 30) such as with a medical grade epoxy, preferably highly translucent to the selected radiation. The reflective sheath 57 can be formed from, for example, a flexible polymer coated with highly reflective material such as, for example, a thin metallic or painted coating such as with a gold or silver base.

Referring to FIG. 8D, in an embodiment, a catheter instrument 325 includes a reflective sheath 57 that is attached to the balloon 30 by an adhesive 105 and, in an embodiment, fibers 40 are attached to sheath 57 by an adhesive 115 and to balloon 30 by an adhesive 125. Adhesive 105, 115, and 125 can be of a type suitable for catheter applications including, for example, ultraviolet light cured adhesive that is translucent to the appropriate wavelength range(s). A ring element, e.g., such as ring element 90 as shown in FIG. 8A, can be omitted in this embodiment as the adhesives 115 and 125 can distribute fibers 40 about the inner periphery of balloon 30 in the desired manner.

In an embodiment, neither fiber sheath 57 or balloon 30 is fixedly attached to fiber ends 45 but fiber sheath 57 and balloon 30 are attached to each other (as separate components or formed from a single component) to form a pouch-like area in which to hold fiber ends 45. Fibers 40 can then slide within the intervening area between fiber sheath 57 and balloon 30, thus potentially reducing stress caused by balloon 30 and sheath 57 on fibers 40 during balloon expansion.

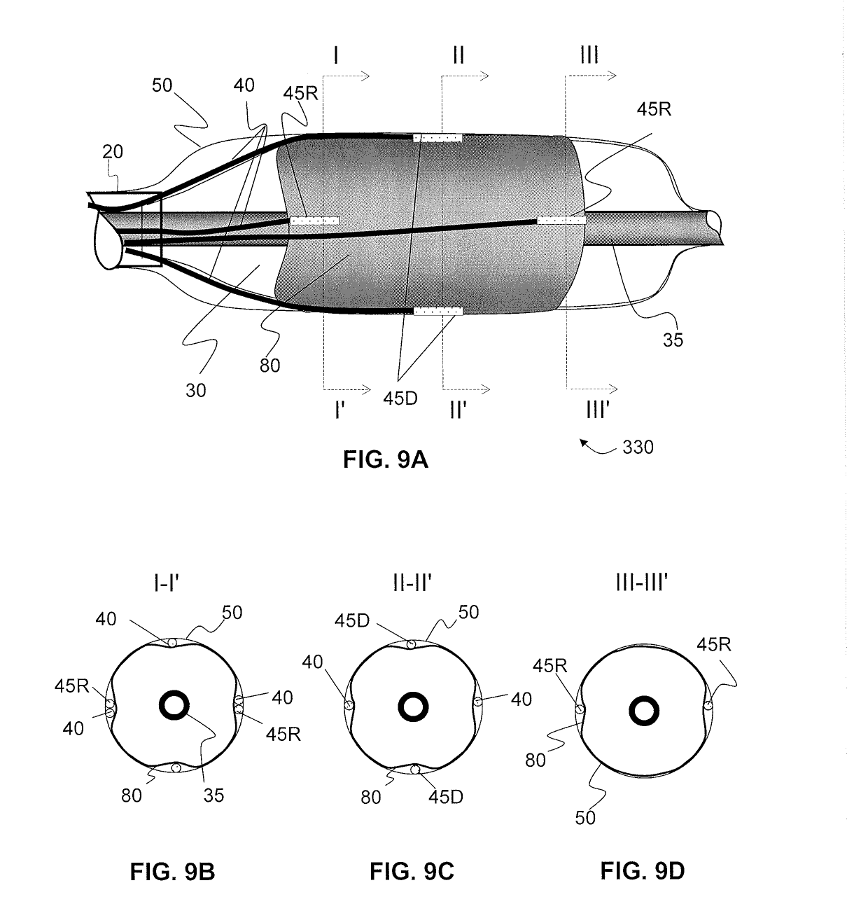

FIG. 9A is an expanded illustrative view of the treatment end of a catheter instrument 330 according to another embodiment of the present invention. FIG. 9B is a cross-sectional view of the catheter of FIG. 9A, taken along section lines I-I′ of FIG. 9A. FIG. 9C is a cross-sectional view of the catheter of FIG. 9A, taken along section lines II-II′ of FIG. 9A. FIG. 9D is a cross-sectional view of the catheter of FIG. 9A, taken along section lines II-II′ of FIG. 9A. Two of the fibers 40 have delivery ends 45D longitudinally separated from the collection ends 45R of four other fibers 40. Two collection ends 45R are also longitudinally separated from two other collection ends 45R. The longitudinal delivery/collection separations can increase the longitudinal breadth of radiation collected from an adjacent vessel and also allow for differentiation between signals collected at longitudinally distinct portions of an adjacent vessel. In an embodiment, all of the six fibers 40 have a core diameter of about 50 microns or less. In various other embodiments, the relative longitudinal positions and separations of collection ends 45R and 45D can be adapted for distinguishing signals associated with particular distinct longitudinal and circumferential sections of an outer lumen wall.

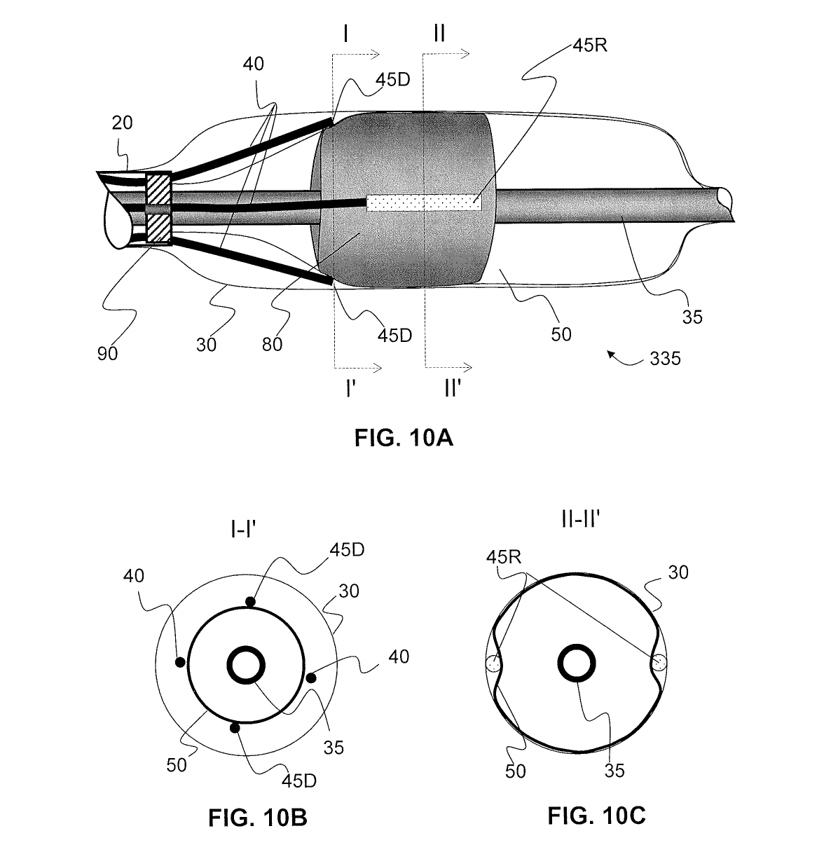

FIG. 10A is an expanded illustrative view of the treatment end of a catheter instrument 335 according to another embodiment of the present invention. FIG. 10B is a cross-sectional view of the catheter of FIG. 10A, taken along section lines I-I′ of FIG. 10A. FIG. 10C is a cross-sectional view of the catheter of FIG. 10A, taken along section lines II-II′ of FIG. 10A. In this embodiment, fiber delivery ends 45D of fibers 40 terminate at a longitudinally separated proximate position in relation to fiber receiving ends 45R. In an embodiment, the delivery ends 45D of fibers 40 terminate so that a substantial amount of light that is delivered includes a more pronounced longitudinal component directed from the proximate to the distal ends of inner balloon 50 (and toward receiving ends 45R). In an embodiment, receiving ends 45R are cleaved without further modification such as with the use of a common fiber cleaving tool. As described above in reference to several other embodiments, an interior balloon 30 can be used to inflate the lumen and push the ends of fibers 40 out toward the inner periphery of the outer covering/balloon 30 and proximate to a lumen wall (not shown). In an embodiment, a reflective surface 80 (such as that previously referenced) on inner balloon 50 is included for improving the delivery and collection of light about the outer covering/balloon 30.

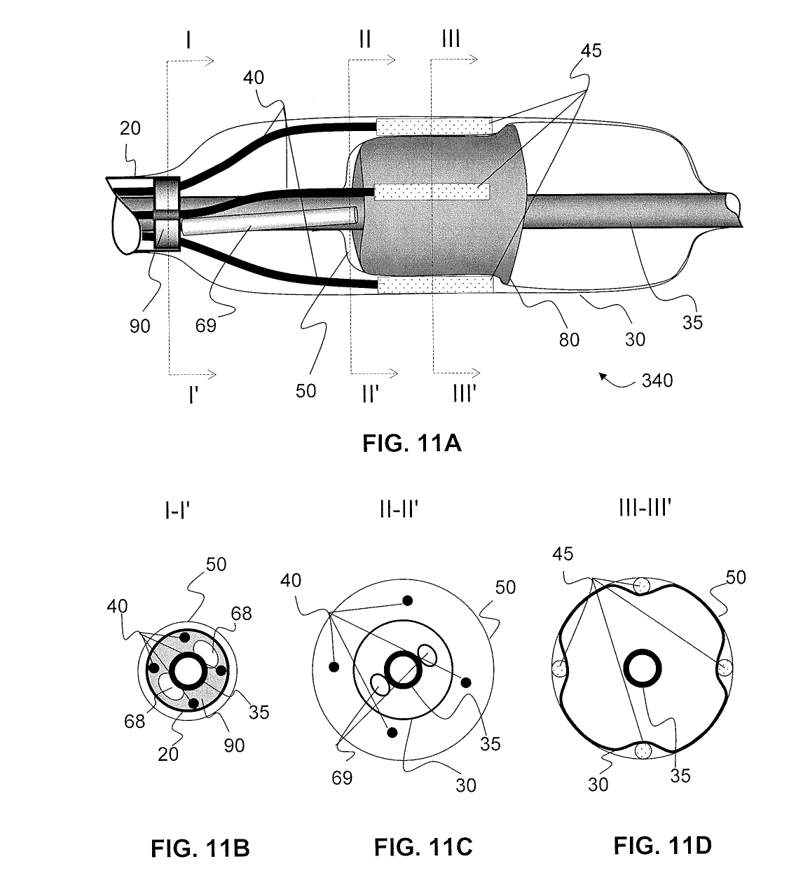

FIG. 11A is an expanded illustrative view of the treatment end of a catheter instrument 340 according to another embodiment of the present invention. FIG. 11B is a cross-sectional view of the catheter of FIG. 11A, taken along section lines I-I′ of FIG. 11A. FIG. 11C is a cross-sectional view of the catheter of FIG. 11A, taken along section lines II-II′ of FIG. 11A. FIG. 11D is a cross-sectional view of the catheter of FIG. 11A, taken along section lines III-III′ of FIG. 11A. In this embodiment, inner balloon 50 is significantly shorter than outer covering/balloon 30 and has its proximate end significantly distal to the proximate end of outer covering/balloon 30. Flush lumen extensions 69 transfer fluid to and from widened openings 68 within a ring 90 and inner balloon 50. In an embodiment, inner balloon 50 is not completely sealed with respect to outer covering/balloon 30 and includes a small opening. For example, in an embodiment, lumen extensions 69 are not fully engaged/sealed over widened openings 68 such that when fluid is supplied through openings 68 to inner balloon 50, fluid media is also supplied (less rapidly) to outer covering/balloon 30.

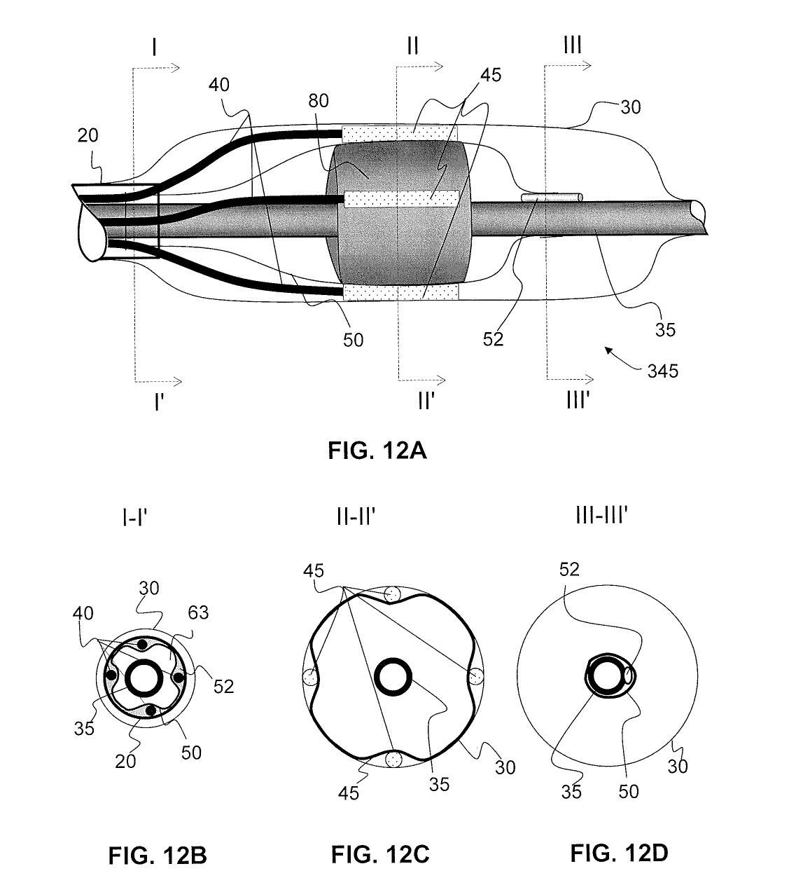

FIG. 12A is an expanded illustrative view of the treatment end of a catheter instrument 345 according to another embodiment of the present invention. FIG. 12B is a cross-sectional view of the catheter of FIG. 12A, taken along section lines I-I′ of FIG. 12A. FIG. 12C is a cross-sectional view of the catheter of FIG. 12A, taken along section lines II-II′ of FIG. 12A. FIG. 12D is a cross-sectional view of the catheter of FIG. 12A, taken along section lines III-III′ of FIG. 12A. In another embodiment, inside balloon 50 includes a secondary flush port 52 such that as inside balloon 50 is filled with fluid through port 63, fluid flows into and also less rapidly fills outside covering/balloon 30.

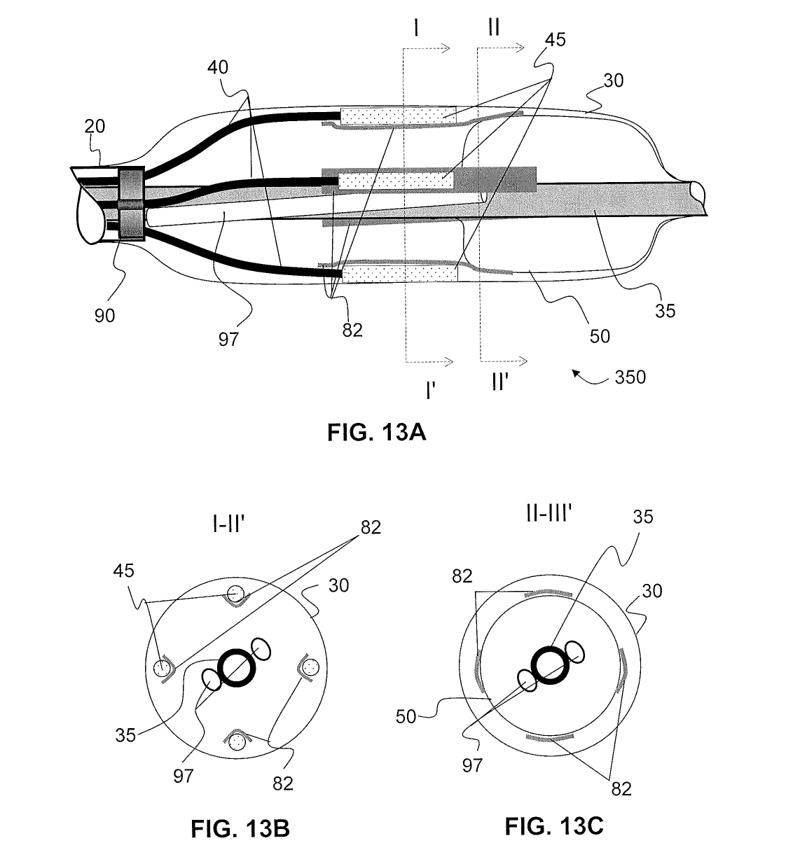

FIG. 13A is an expanded illustrative view of the treatment end of a catheter instrument according to another embodiment of the present invention. FIG. 13B is a cross-sectional view of the catheter of FIG. 13A, taken along section lines I-I′ of FIG. 13A. FIG. 13C is a cross-sectional view of the catheter of FIG. 13A, taken along section lines II-II′ of FIG. 13A. In an embodiment and as an alternative to a reflective surface 80 over inside balloon 50, solid elastic reflective elements 82 are attached to inside balloon 50. Fibers 40 are attached at their inside edges to the reflective elements 82. As balloon 50 is expanded, reflective elements 82 and attached fiber ends 45 remain proximate to the inside surface of outside covering/balloon 30. Fiber ends 45 can be attached to separate reflective elements 82 in a manner, for example, similar to the attachment of fiber ends 45 to the common reflecting element 57 of FIGS. 8A-8C. Reflecting elements 82 can be formed, for example, out of thin reflective metallic strips or plastic pieces coated with reflective material. In an embodiment, a flush lumen extension 69 extends from a ring element 97 in a manner similar to that shown and described in reference to FIGS. 11A-11D.

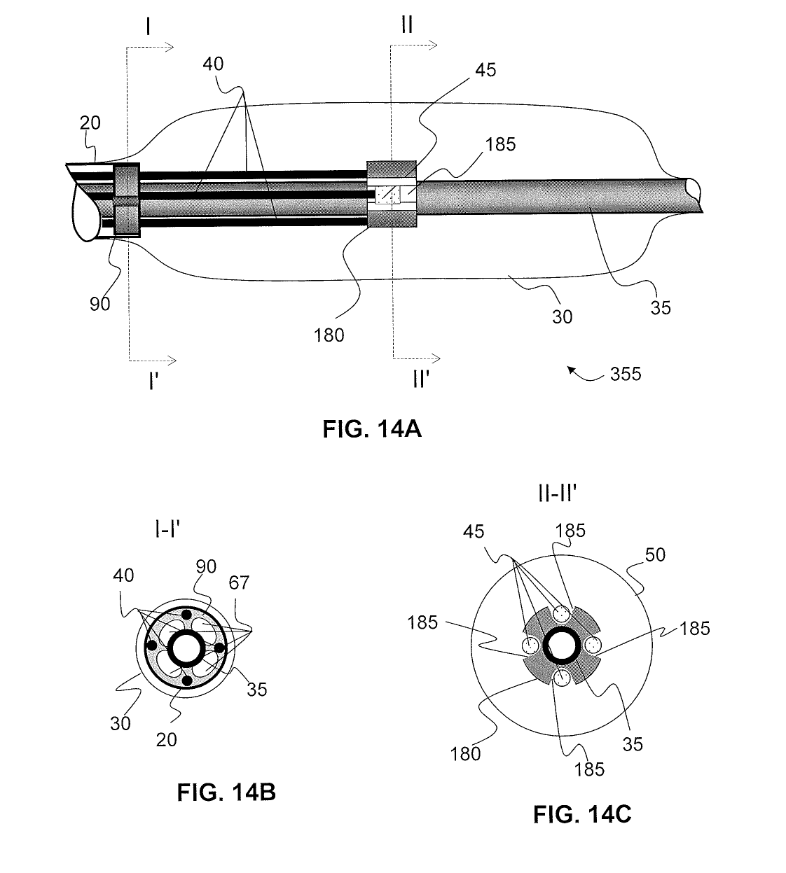

FIG. 14A is an expanded illustrative view of the treatment end of a catheter instrument 355 according to another embodiment of the present invention. FIG. 14B is a cross-sectional view of the catheter of FIG. 14A, taken along section lines I-I′ of FIG. 14A. FIG. 14C is a cross-sectional view of the catheter of FIG. 14A, taken along section lines II-II′ of FIG. 14A. In this embodiment, fibers 40 are fixed contiguously to catheter sheath 20 and guidewire lumen 35. A reflecting element 180 is formed about guidewire lumen 35 having shaped reflective surfaces 185 that can help distribute or collect light to or from an area generally concentrated across an adjacent lumen (not shown). In an embodiment, reflective surfaces 185 are parabolic and shaped so that adequate light travels to an adjacent lumen through an end 45 designated for light delivery and light is returned from the lumen to an end 45 designated for light collection. The shape of the parabola can be optimized based on the size and distribution/collection profile of fiber ends 45 and the estimated distance between distribution/collection ends 45 from each other and from the lumen wall (or the outside of outer balloon 30).

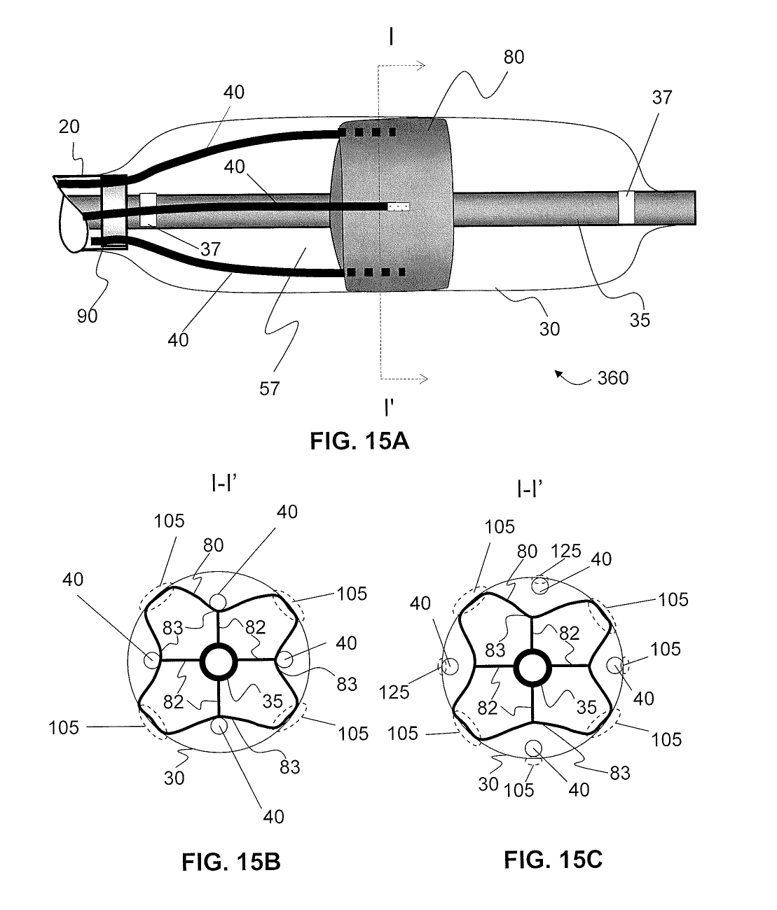

FIG. 15A is an expanded illustrative view of the treatment end of a catheter instrument 360 according to another embodiment of the invention. FIG. 15B is a cross-sectional view of the catheter of FIG. 15A, taken along section lines I-I′ of FIG. 15A. FIG. 15C is an alternative embodiment of a cross-sectional view of the catheter of FIG. 15A, taken along section lines I-I′ of FIG. 15A. Reflective surface 80 can be attached at points along the inside of a balloon 30 by an adhesive 105 wherein the attachment points are circumferentially offset from fibers 40. Reflective surface 80 is also tethered to a guidewire sheath 35 by connector sections 82 at points generally circumferentially aligned with fibers 40. The connector section 82 can be of a predetermined radial length and stiffness so that when balloon 80 is in an expanded state, fibers 40 are held along a section 83 of reflective surface 80 that is recessed from the surface of balloon 30. Recessing fibers 40 from the outside surface of balloon 30 can, for example, decrease the occurrence of radiation being blocked by a stent positioned around balloon 30. Referring to FIG. 15C, fibers 40 can alternatively be attached to balloon 30 by an adhesive 125 as in, for example, described in reference to FIG. 8D. Offsetting the reflector 57 from fibers 40 can, for example, increase the scope of delivered and/or collected radiation incident upon reflector 57.

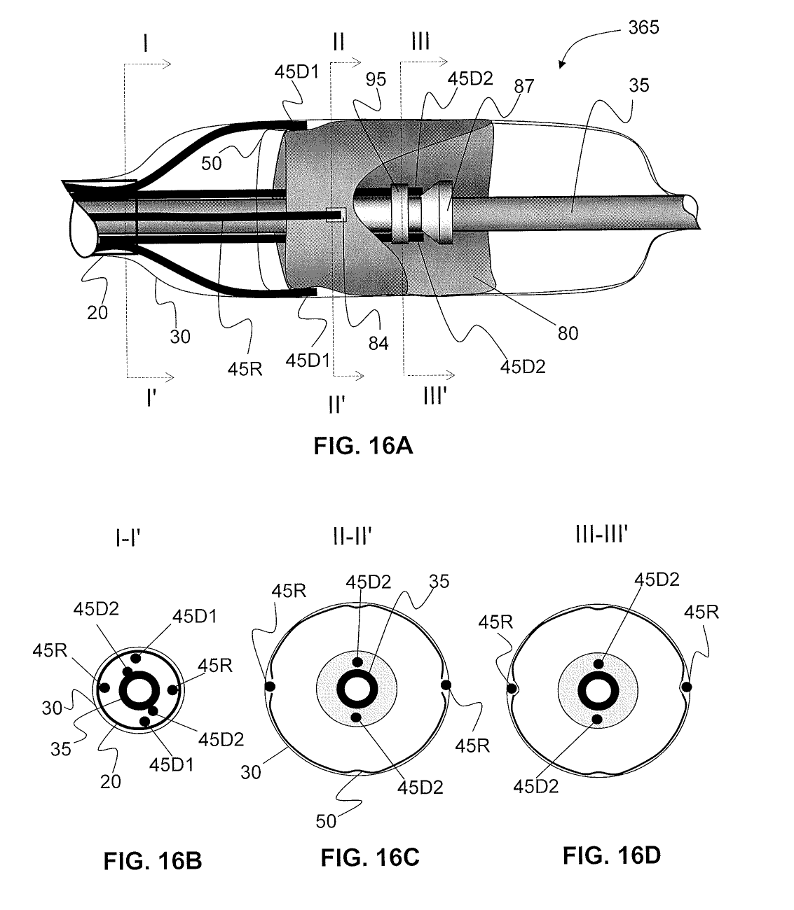

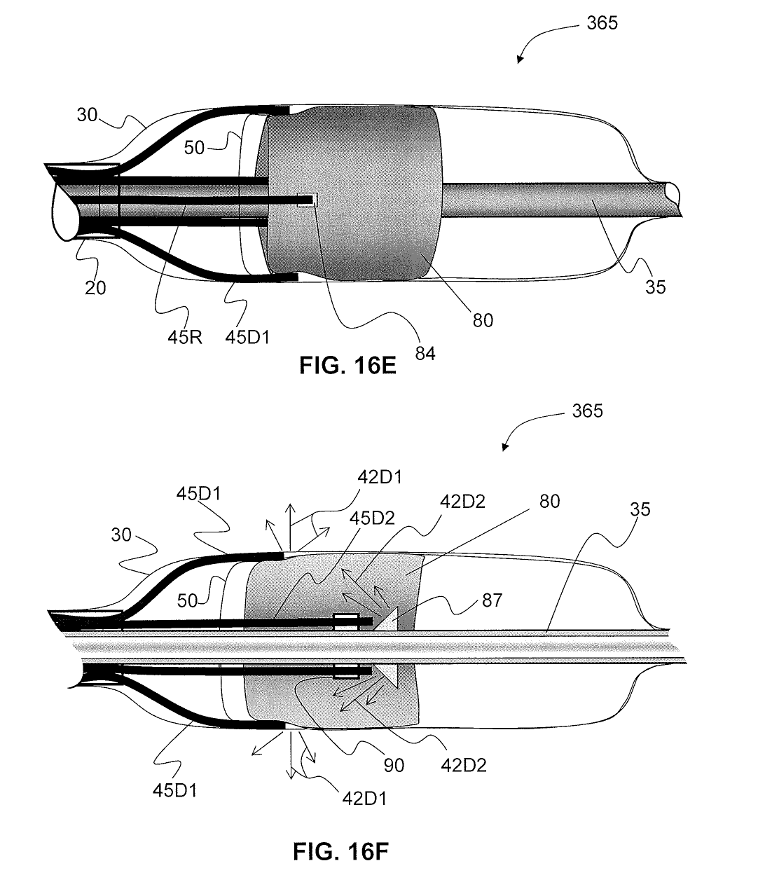

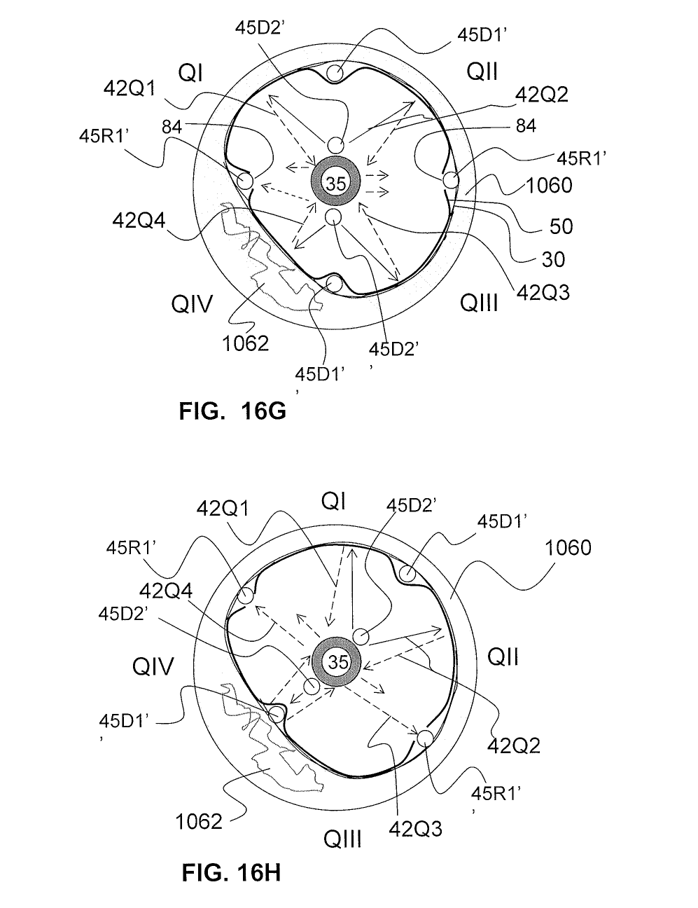

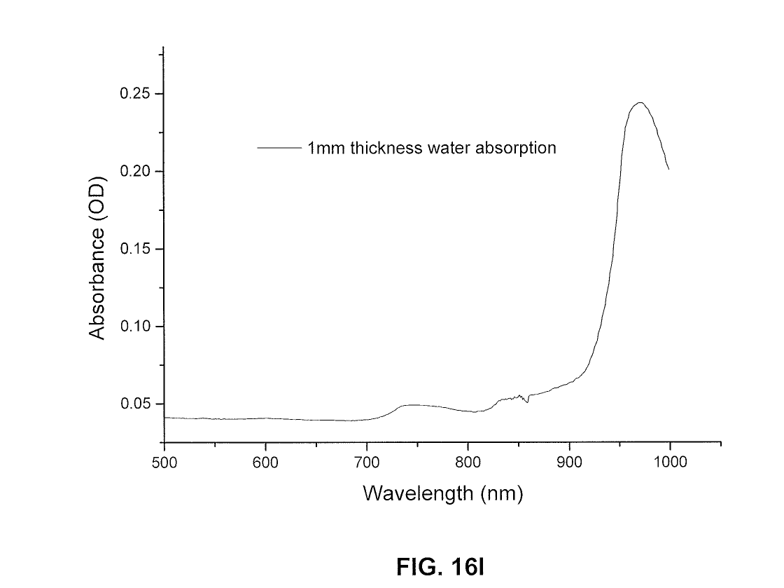

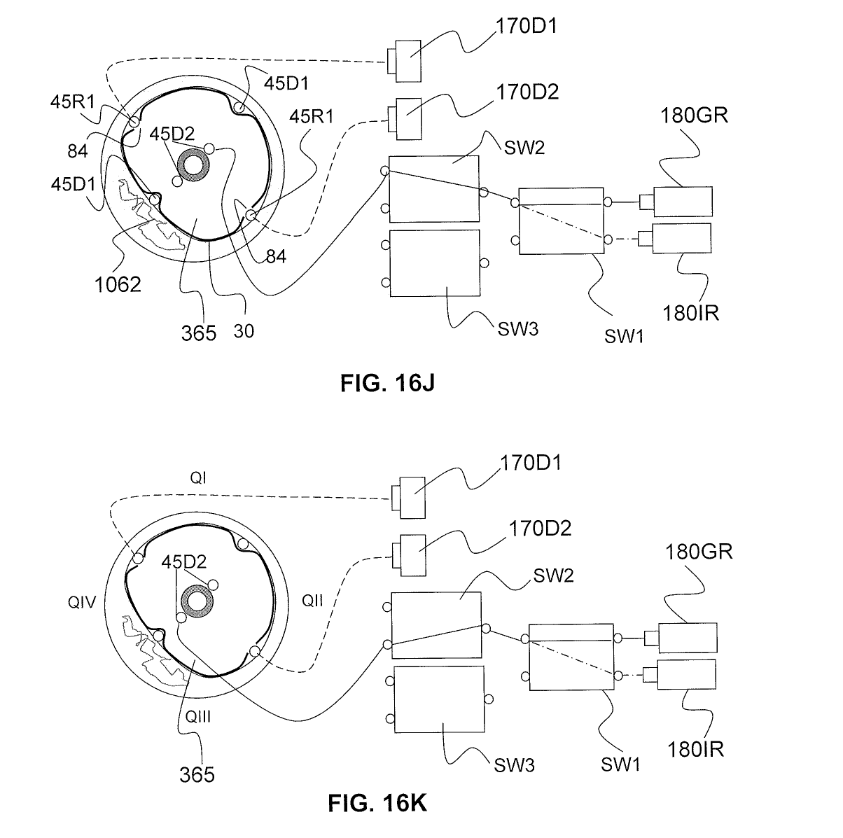

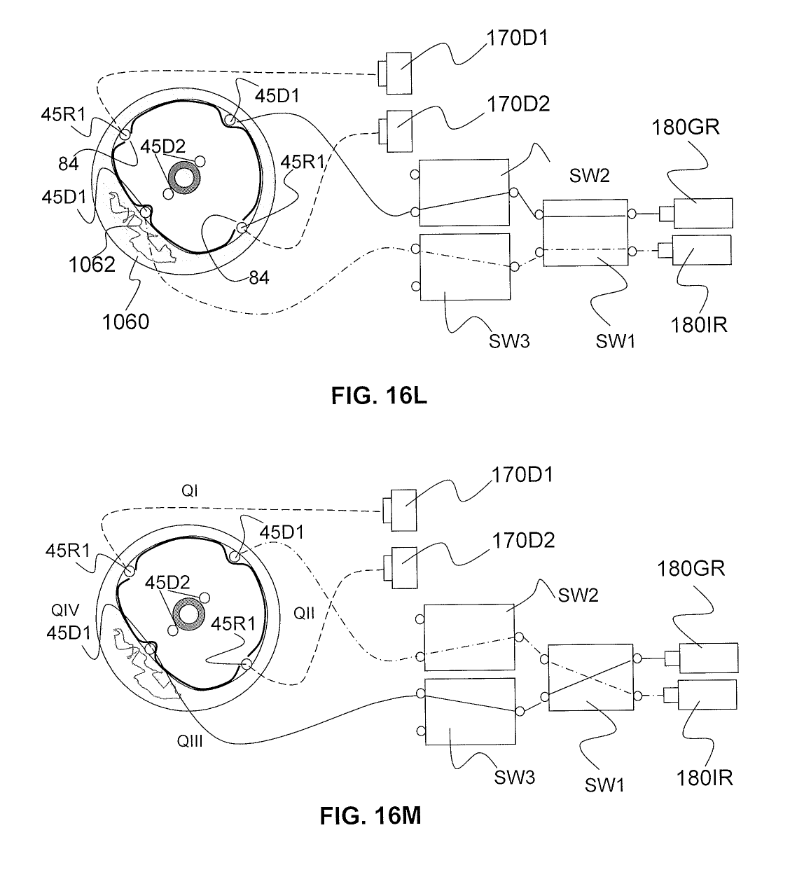

FIG. 16A is an expanded illustrative view of the treatment end of a catheter instrument 365 according to another embodiment of the present invention. FIG. 16B is a cross-sectional view of the catheter of FIG. 16A, taken along section lines I-I′ of FIG. 16A. FIG. 16C is a cross-sectional view of the catheter of FIG. 16A, taken along section lines II-II′ of FIG. 16A. FIG. 16D is a cross-sectional view of the catheter of FIG. 16A, taken along section lines III-III′ of FIG. 16A. FIG. 16E is another expanded illustrative view of the treatment end of the catheter of FIG. 16. FIG. 16F is an expanded illustrative cutout view of the catheter of FIG. 16A. In an embodiment, delivery fibers 45D1 and collection fibers 45R are held between an interior balloon 50 with a reflective surface 80 and an exterior balloon 30. In an embodiment, the fibers 45D1 and 45R are affixed with an adhesive to balloon 50. Delivery fibers 45D2 are held fixed contiguously to catheter sheath 20 and guidewire lumen 35 by a ring element 95. A cone-shaped reflecting element 87 is arranged to distribute radiation from delivery fibers 45D2 to collection fibers 45R through a window 84 in the reflective surface 80. In an embodiment, and as further described below in reference to FIGS. 16G and 16H, signals between delivery fibers can be used to determine whether balloon 30 is fully expanded. Balloon 30 is attached by its proximate end to a catheter sheath 20 and by its distal end to a guidewire lumen 35.

FIGS. 16G and 16H are illustrative cross-sectional views of the catheter instrument 365 of FIG. 16A within a lumen 1060. In an embodiment, a circumference of the surface of balloon 30 is demarcated by four quadrants Q1, QII, QIII, and QIV. In an embodiment, signals from delivery fibers 45D2′ and 45D2″ (contiguous with catheter 365) to collection fibers 45R1′ and 45R1″ are used to compare the relative proximity of the surface of balloon 30 along each of the circumferential quadrants QI-QIV. If signals associated with one or more of the quadrants is sufficiently disproportionate to signals associated with one or more of the other quadrants, this may be an indication that the balloon 30 is not fully inflated and requires additional inflation. These signals together with signals received in response to light delivered by fibers 45D1′ and 45D1″ about the balloon for example, as described further herein relating to detecting the presence of blood and plaque, can further indicate whether the balloon is mal-apposed and/or underinflated.

For example, signals between a delivery fiber 45D2′ and a collection fiber 45R1′, such as along exemplary trace lines 42QI, can be used to compare the relative proximity that the surface of balloon 50 has to the center of the catheter along quadrant QI in relation to the other balloon surface quadrants' proximity (i.e., in comparison to signals such as along exemplary trace lines 42QII, 42QIII, and 42QIV). Referring to FIG. 16G, where a region 1062 of partial blockage is preventing balloons 30 and 50 from fully expanding, stronger signals associated with the relative positioning of quadrant QIV of the balloon 30 indicate that quadrant QIV is not as fully expanded as the other quadrants QI, QII, and QIII.

In an embodiment, diffuse reflectance spectroscopy is employed between wavelengths of 250 and 2500. In an embodiment, ratios between the absorbance signals of two or more wavelengths are used to indicate a relative proximity of the balloon surface. In an embodiment, one of the two or more wavelengths is between about 250 and 750 nanometers and another of the two or more wavelengths is between about 800 and 1000 nanometers. In an embodiment, one of the two or more wavelengths is green visible light (or about 520 nanometers) and one of the two or more wavelengths is about 800 nanometers or about 980 nanometers, wavelengths that will generally be more sensitive to the presence of water and blood. FIG. 16I is a chart of absorption measurements comparing radiation at various wavelengths traveling through water across a 1 mm span (a span which is typical of the distance that light travels according to various embodiments described herein). As can be seen, a wavelength of about 980 nanometers provides a high degree of sensitivity for this span of travel.

FIG. 16J is an illustrative schematic of an optical source and detector configuration during a step of the operation of the catheter of FIG. 16A according to an embodiment of the invention. In an embodiment, a source 180GR provides green visible radiation, e.g., about 520 nanometers, and source 180IR provides near-infrared radiation, e.g., about 800 or 980 nanometers. Three optical switches SW1, SW2, and SW3 direct radiation from the sources 180GR and 180IR to the various delivery fibers, including fibers 45D1 and 45D2.

In an embodiment, an initial optical configuration as shown in FIG. 16J directs radiation from one or both of the sources 180GR and 180IR to one of the 45D2 delivery fibers so as to illuminate two adjacent circumferential quadrants, e.g., QI-QIV (see FIGS. 16G & 16H and accompanying description), through which radiation is delivered to collection fibers 45R1 and analyzed.

FIG. 16K is an illustrative schematic of an optical source and detector configuration during another step of the operation of the catheter of FIG. 16A according to an embodiment of the invention. In another step of the operation of catheter 365, the two circumferential quadrants, e.g., of QI-QIV of those not illuminated as in FIG. 16J, are then illuminated and analyzed by switching the delivery radiation from one or both sources 180IR and 180GR to the other fiber 45D2. Delivery of the different types of radiation can be performed simultaneously or, in an embodiment, at separate times.

FIG. 16L is an illustrative schematic of an optical source and detector configuration during another step of the operation of the catheter of FIG. 16A according to an embodiment of the invention. In another step of the operation of catheter 365, delivery fibers 45D1 deliver radiation to areas about the periphery of balloon 30 including the walls of lumen 1060. Radiation from the lumen wall is then collected by collection fibers 45R1 and analyzed such as in accordance with various embodiments referred to herein.

FIG. 16M is an illustrative schematic of an optical source and detector configuration during another step of the operation of the catheter of FIG. 16A according to an embodiment of the invention. In another step of the operation of catheter 365, the two circumferential quadrants, e.g., of QI-QIV of those not illuminated as in FIG. 16L, are then illuminated and analyzed by swapping the delivery radiation from sources 180IR and 180GR between delivery fibers 45D1. Delivery of the different types of radiation can be performed simultaneously or, in an embodiment, at separate times.

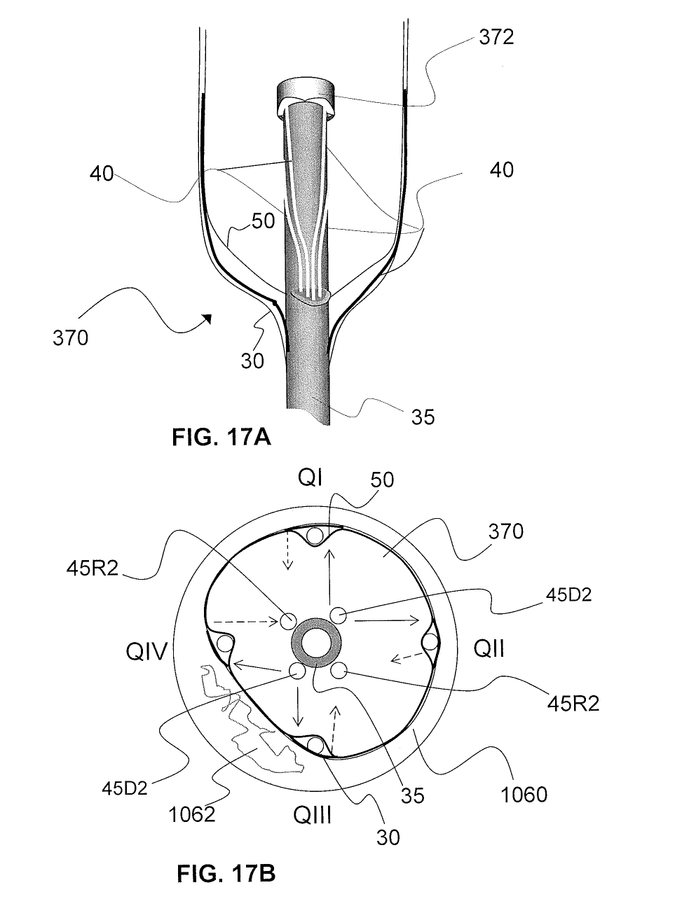

FIG. 17A is an illustrative schematic of another embodiment of a catheter configuration 370 including two delivery fibers and two collection fibers contiguous with the guidewire sheath for detecting balloon underexpansion. FIG. 17B is an illustrative cross-sectional schematic of the delivery fibers 45D2 and collection fibers 45R2 positioned for analyzing the expansion profile of the balloons of FIG. 17A within a lumen 1060. In an embodiment, a multi-faceted reflecting element 372 is positioned so as to deliver and receive radiation about the interior of balloon 50. A region of blockage 1062 causes balloons 30 and 50 to be initially underinflated about circumferential regions QIII and QIV, causing received signals correlating to those regions to be stronger than signals correlation to the other regions QI and QII. Various embodiments of reflective elements can be used such as those described in U.S. patent application Ser. No. 11/834,096, published as U.S. Patent Application Publication No. US 2007/0270717 A1, the entire contents of which is herein incorporated by reference.

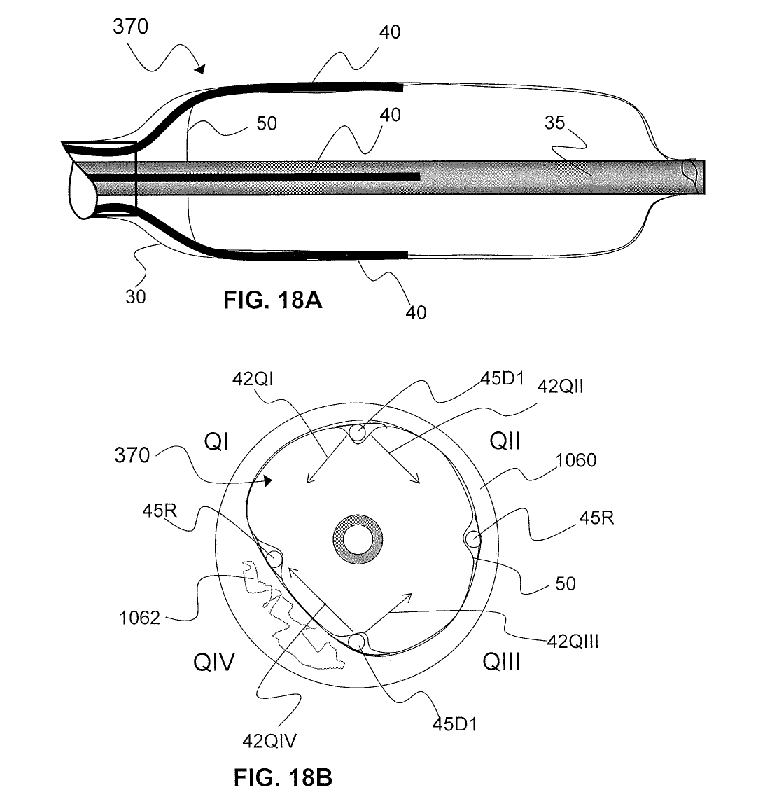

FIG. 18A is an illustrative schematic of another embodiment of a catheter configuration 370 including two delivery fibers and two collection fibers of fibers 40 positioned along the inner surface of the balloon 30. FIG. 18B is an illustrative cross-sectional schematic of the delivery fibers 45D1 and collection fibers 45R positioned for analyzing the expansion profile of the balloons of FIG. 18A within a lumen 1060. Delivery fibers 45D1 and collection fibers 45R are held between an inner balloon 50 and an outer balloon 30 such as described in reference to other embodiments included herein. The surfaces of inner balloon 50 are translucent to radiation delivered by delivery fibers 45D1, allowing signals 42QI, 42QII, etc. to travel to collection fibers 45R and be analyzed in order to determine the relative positioning and expansion/under-expansion of each of the circumferential regions QI-QIV. For example, as illustrated in FIG. 18B, the signals 42QIV travel a shorter distance from a delivery fiber to a collection fiber than do the other signals, thus indicating that the circumferential region QIV is under-expanded relative to the other circumferential regions.

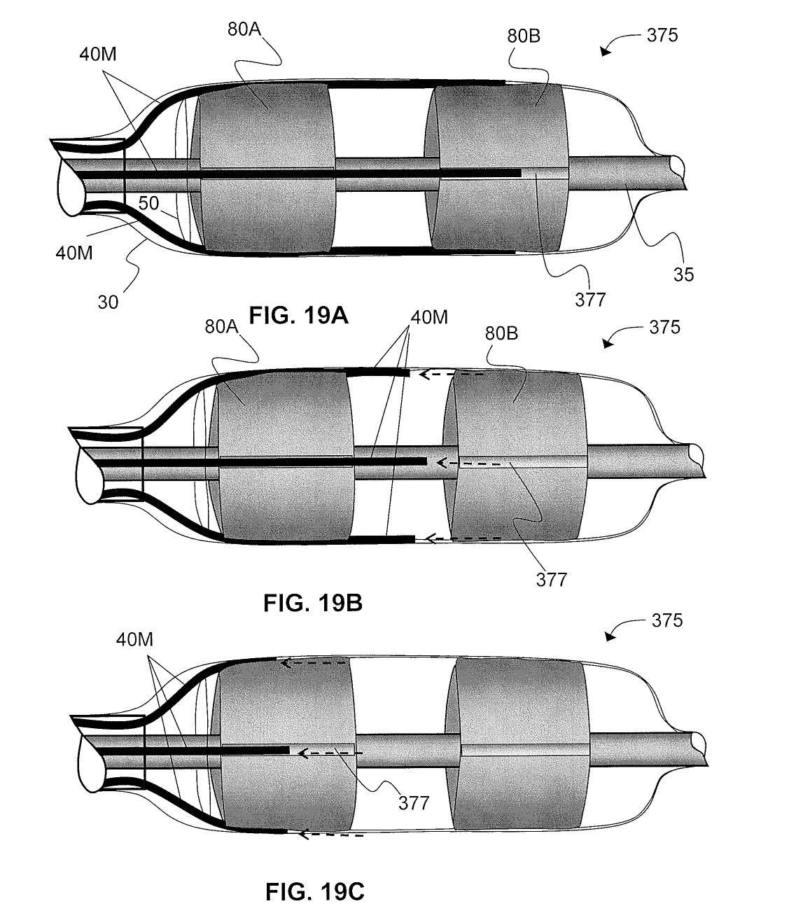

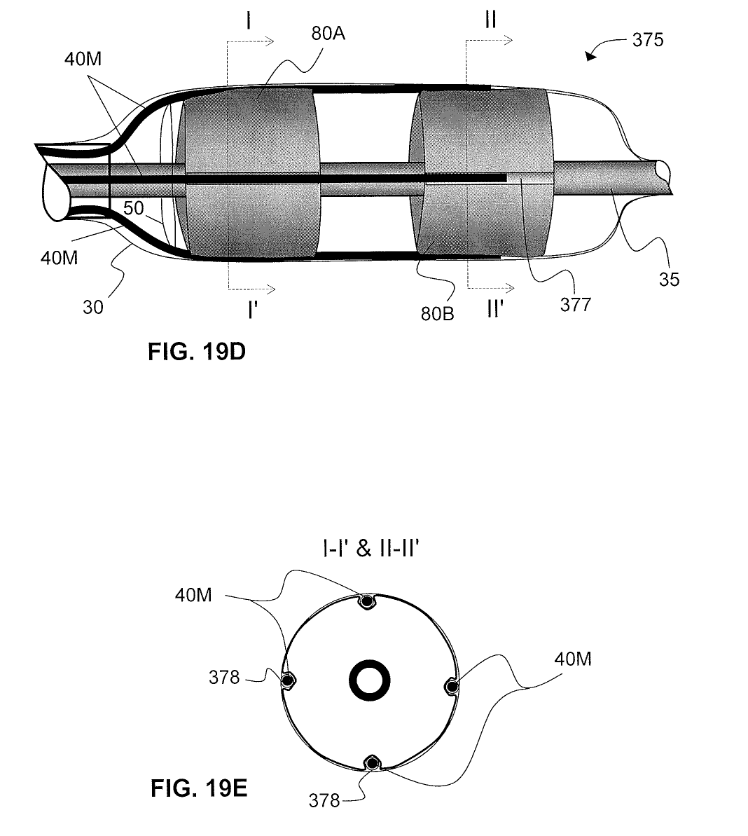

FIGS. 19A and 19D are illustrative views of the treatment end of a catheter instrument 375 with slidably movable fibers according to an embodiment of the present invention. FIG. 19B is an illustrative view of the treatment end of the catheter instrument 375 of FIG. 19A with fibers 40M moved near the longitudinal center of balloon 30. FIG. 19C is an illustrative view of the treatment end of the catheter instrument 375 of FIG. 19A with fibers 40M positioned near the proximal end of balloon 30. FIG. 19E is a cross-sectional view of the catheter of FIG. 19D, taken along section lines I-I′ and II-II′ of FIG. 19D. In an embodiment, the slidably movable fibers 40M can be shifted to various positions along balloon 30 so as to provide more complete and/or detailed analysis at various positions along balloon 30. Reflective surfaces 80A and 80B include grooved sections 377 (shown in cross section within FIG. 19E) within which fibers 40M can slide. In an embodiment, translucent nylon sleeves 378 surround fibers 40M within which they can slide. Sleeves 378 can also be directly attached to inner balloon 50 while permitting the movement of fibers 40M within. In an embodiment, a procedure includes first positioning fibers 40M along a distal portion of balloon 30 overlapping a reflective surface 80B as shown in FIG. 19A. Analysis can be performed about the exterior of balloon 30 as described in various embodiments herein for purposes of determining the content of the lumen wall and/or proper apposition of balloon 30 against the lumen wall. Fibers 40M can then be moved to another position such as near the longitudinal center of balloon 30 and between and unblocked overlapping reflective surfaces 80A and 80B. At such a position where the fibers can deliver or collect light to or from the interior of the balloon, analysis of the shape of balloon 30 can be performed such as in accordance with various embodiments described herein, for example, the embodiments described in reference to FIGS. 18A and 18B. Fibers 40M can then be positioned along reflective surface 80A and analysis performed about the proximal end of balloon 30. In an embodiment, as many as six positions along balloon 30 are analyzed in about twenty seconds or less.

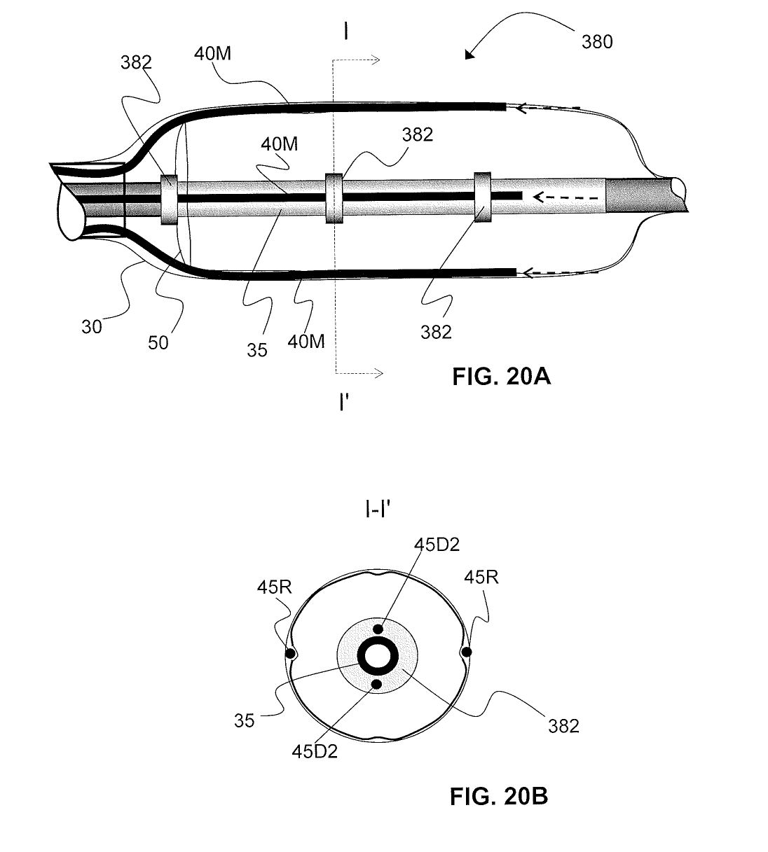

FIG. 20A is an illustrative view of the treatment end of a catheter instrument 380 with slidably movable fibers according to another embodiment of the present invention. FIG. 20B is a cross-sectional view of the catheter of FIG. 20A, taken along section lines I-I′ of FIG. 20A. In an embodiment, the distal ends of two slidable collection fibers of fibers 40M are positioned to be adjacent the periphery of balloons 30 and 50 and two slidable delivery fibers of fibers 40M are positioned and remain contiguous to the guidewire sheath 35 by being held and longitudinally slidable within rings 382. In an embodiment, the catheter 380 can function in a manner such as described in FIGS. 16G and 16H while having fibers 40M moved along various positions along catheter instrument 380, providing a more complete analysis along balloon 30.

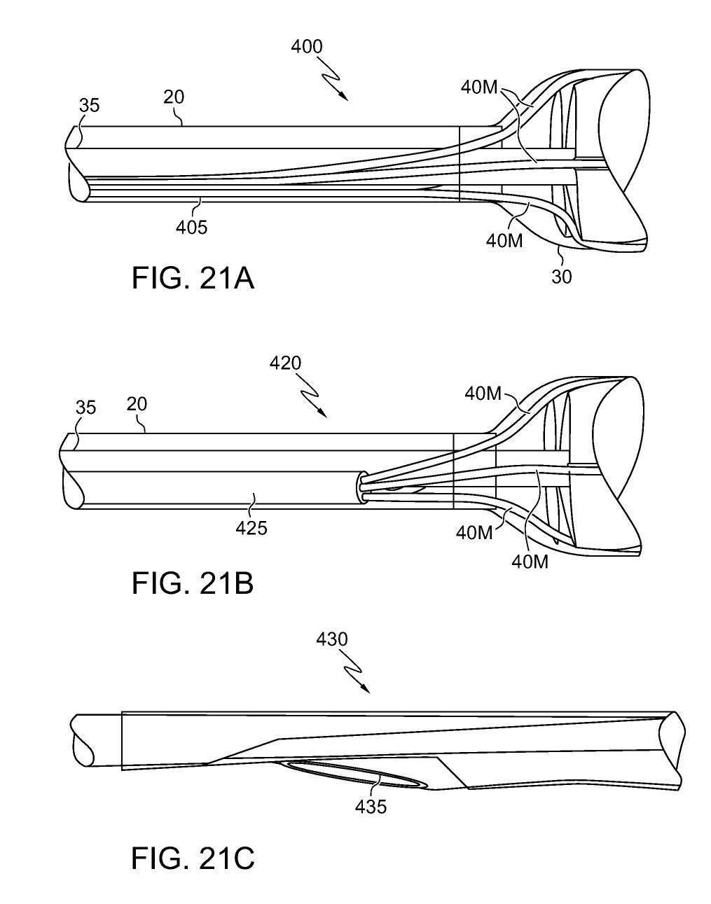

FIG. 21A is another illustrative view of an arrangement of slidably movable fibers 40M integrated with an inflatable balloon catheter 400. In an embodiment, the fibers 40M are adhered together at a location 405 within catheter body 20 so that they may slidably move together.

FIG. 21B is another illustrative view of an arrangement of slidably movable fibers integrated with an inflatable balloon. In an embodiment, slidable fibers 40M are tethered together by an outer covering 425. The covering can be, for example, polymid or another polymer.

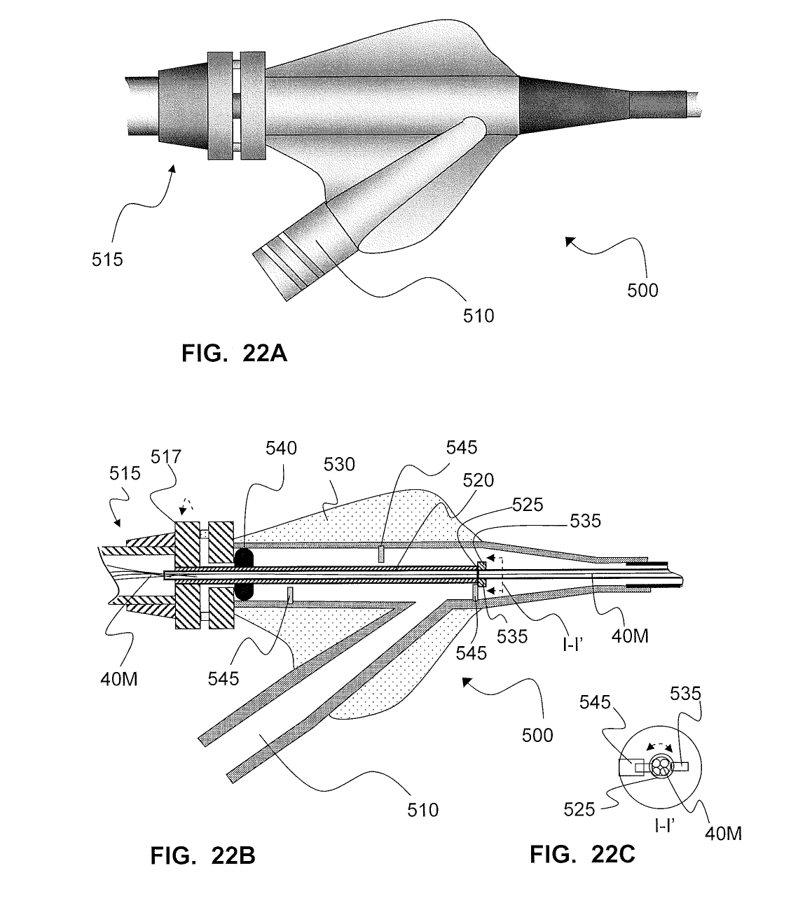

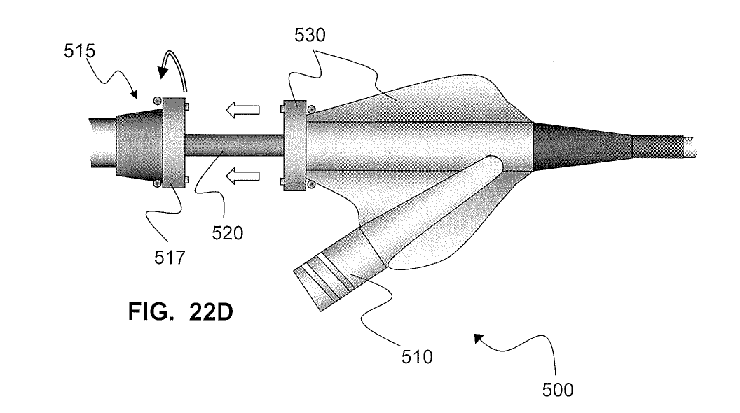

FIG. 21C is an illustrative view of a section of a catheter 430 having guidewire lumen opening 435 according to an embodiment of the invention. In an embodiment, guidewire lumen opening 435 is located near the distal end of catheter 430 for rapid catheter exchange as understood by one of ordinary skill in the art. FIG. 22A is an illustrative view of the proximate end of a catheter instrument 500 for manipulating slidable fibers according to an embodiment of the invention. FIG. 22B is a cross-sectional illustrative view of the catheter instrument 500 of FIG. 22A. FIG. 22C is an illustrative cross-sectional view of the catheter instrument of FIGS. 22A-B across section lines I-I′ of FIG. 22B. FIG. 22D is an illustrative view of proximate end the catheter instrument 500 with a slidably movable section 515 in an open position. In an embodiment, slidably movable section 515 is included for repositioning fibers 40M such as within the catheter components described in connection with FIGS. 19A-19C, 20A-B, and 21A-C. Section 515 includes an elongate tubular piece 520 that is fixedly connected to fibers 40M such as with adhesive and/or a clamp 525. The remaining components of the catheter 500 remain while a slidable section 515 may be pulled/released to draw fibers 40M toward the proximate end of the catheter 500. The elongate tubular piece 520 remains within segment 530 and a gasket 540 prevents fluid (e.g., balloon expansion media) from exiting through the interface between segments 530 and 515. In an embodiment, catches 535 (attached to tubular piece 520) and 545 (attached to segment 515) can prevent segment 515 (including tubular piece 520) from sliding. In an embodiment, a handle 517 can rotate segment 515 and tubular piece 520 so as to disengage catches 535 and 545 and allow segment 515 to slide. In an embodiment, catches 545 are distributed along segment 530 so that when segment 515 is disengaged from a catch 545 and segment 515 proceeds to slide, another catch 545 positioned further toward the proximate end of the catheter will engage a catch 535 and stop the progress of sliding motion until handle 517 is rotated again. In an embodiment, catches 545 are also distributed so that the catch points correspond to predetermined longitudinal positions of fibers 40M along a balloon component (e.g., as shown in FIGS. 19A-C and 20A-B). Pressure from fluid media entering through a port 510 may also apply pressure on segment 515 so that segment 515 slides proximately when catches 535 and 545 are not engaged.

It will be understood by those with knowledge in related fields that uses of alternate or varied materials and modifications to the methods disclosed are apparent. This disclosure is intended to cover these and other variations, uses, or other departures from the specific embodiments as come within the art to which the invention pertains.

What is claimed is:

1. A catheter for placement within a body lumen, the catheter comprising:
a flexible conduit that is elongated along a longitudinal axis, the flexible conduit having a proximal end and a distal end;
at least one delivery waveguide and at least one collection waveguide extending along the flexible conduit, the at least one delivery waveguide and the at least one collection waveguide constructed and arranged to transmit radiation at a wavelength in a range of about 250 to 2500 nanometers;
a transmission output of the at least one delivery waveguide and a transmission input of the at least one collection waveguide;
a flexible, expandable first surface surrounding a segment of the conduit, said transmission output and said transmission input located within said flexible, expandable first surface; and
a second surface radially translatable with respect to said flexible, expandable first surface, said at least one transmission input located between a portion of said flexible, expandable first surface and a portion of the second surface.

2. The catheter of claim 1 wherein at least one of said first surface and said second surface forms a surface of a lumen-expanding balloon.

3. The catheter of claim 1 wherein the at least one of the delivery and collection waveguides comprises at least one optical fiber and wherein the longitudinal axis of a tip of said at least one optical fiber is arranged to be substantially parallel with said first surface.

4. The catheter of claim 3 wherein a recess is formed out of said tip of the at least one optical fiber so as to allow the transmission of radiation in a direction transverse to said longitudinal axis of said tip.

5. The catheter of claim 1 comprising a first conduit for directing inflation media to the interior of the flexible, expandable first surface.

6. The catheter of claim 5 comprising a second conduit for directing inflation media between the flexible, expandable first surface and the second surface.

7. The catheter of claim 6 wherein the first conduit and the second conduit are arranged to initially direct more inflation media to the interior of the flexible, expandable first surface in which inflation media is directed to said area between the flexible, expandable first surface and the second surface.

8. The catheter of claim 7 wherein said first conduit comprises a greater volumetric capacity for transferring fluid than said second conduit.

9. The catheter of claim 5 wherein said first conduit is in direct fluid communication to each of the interior of the flexible, expandable first surface and said area between the flexible, expandable first surface and the second surface.

10. The catheter of claim 1 wherein the second surface comprises a reflective surface.

11. The catheter of claim 10 wherein the reflective surface forms a circumferential band around the flexible conduit.

12. The catheter of claim 10 wherein the reflective surface comprises at least one of a gold-colored and silver-colored coating.

13. The catheter of claim 12 wherein the coating comprises paint.

14. The catheter of claim 10 wherein the reflective surface is applied to the catheter by an ion-assisted deposition method.

15. The catheter of claim 10 wherein the reflective surface is concave with respect to the at least one delivery waveguide and the at least one collection waveguide.

16. The catheter of claim 10 wherein the reflective surface comprises a translucent gap through which light radiation can pass between a transmission input or output located outside the periphery of said reflective surface and an area located within the periphery of said reflective surface.

17. The catheter of claim 1 further comprising one or more additional surfaces translatable with respect to said flexible, expandable first surface and wherein one or more additional transmission outputs or inputs are located between a portion of said flexible expandable first surface and portions of said one or more additional surfaces.

18. The catheter of claim 17 wherein said additional surfaces each comprise a reflective surface.

19. The catheter of claim 17 wherein each of said additional surfaces comprises an eyelet attached to said first surface, wherein at least one waveguide passes through the eyelet.

20. The catheter of claim 17 wherein each of said additional surfaces comprises a reflective element.

21. The catheter of claim 20 wherein each of said additional surfaces is attached to at least one of said at least one delivery waveguide and at least one collection waveguide and wherein each of said additional surfaces is attached to said second surface.

22. The catheter of claim 1 wherein the first surface and the second surface form at least one pocket which holds at least one of the at least one delivery waveguide and the at least one collection waveguide.

23. The catheter of claim 22 wherein said first surface and said second surface are arranged so as to hold the tip of said at least one delivery waveguide and the at least one collection waveguide at a predetermined distance from said first surface when said first surface is fully expanded.

24. The catheter of claim 1 wherein the transmission output of the at least one delivery waveguide and the transmission input of the at least one collection waveguide are arranged to facilitate collection of radiation emitted from tissue of a predetermined scope and depth from the flexible, expandable first surface.

25. The catheter of claim 24 wherein the transmission output of the at least one delivery waveguide and the transmission input of the at least one collection waveguide are spaced apart at a predetermined distance to facilitate the collection of radiation emitted from tissue of said predetermined scope and depth.

26. The catheter of claim 25 wherein the transmission output of the at least one delivery waveguide and the transmission input of the at least one collection waveguide are longitudinally spaced apart at a predetermined distance to facilitate the collection of radiation emitted from tissue of said predetermined scope and depth.

27. The catheter of claim 25 wherein the transmission output of the at least one delivery waveguide and the transmission input of the at least one collection waveguide are circumferentially spaced apart at a predetermined distance to facilitate collection of radiation emitted from tissue of said predetermined scope and depth.

28. The catheter of claim 1 further comprising a waveguide having a transmission input or transmission output that is contiguously retained against said flexible conduit.

29. The catheter of claim 28 wherein the transmission output or transmission input that is contiguously retained against said flexible conduit is arranged to deliver or collect radiation transmitted to or from a waveguide retained against said first surface.

30. The catheter of claim 29 wherein the arrangement to deliver or collect radiation transmitted to or from a waveguide retained against said first surface is configured to provide information including the uniformity of expansion of said flexible, expandable first surface.

31. The catheter of claim 1 wherein at least one waveguide extending along the flexible conduit is slidably movable along the longitudinal axis of said flexible conduit.

32. The catheter of claim 31 wherein the second surface comprises a plurality of circumferential reflective bands distributed about the longitudinal axis of said flexible conduit.

33. The catheter of claim 32 wherein the plurality of circumferential reflective bands comprises two bands, one of said two bands positioned at a proximate end of said first surface and one of said two bands positioned at a distal end of said first surface so as to form a translucent region between said two reflective bands.

34. The catheter of claim 31 comprising a slidably movable handle located at the proximate end of said flexible conduit, the slidably movable handle connected to the at least one slidably movable waveguide so as to allow for slidably moving the at least one slidably movable waveguide.

35. The catheter of claim 34 wherein the slidably movable handle comprises a mechanical locking mechanism for positioning the slidably movable waveguides at predetermined longitudinal positions along said first surface.

36. The catheter of claim 31 wherein each of the at least one slidably movable waveguide is retained in a sleeve within which the at least one slidably movable waveguide can slide.

36. The catheter of claim 36 wherein said sleeve is constructed of a translucent material.

37. A system for probing and treating a body lumen comprising:
a flexible conduit that is elongated along a longitudinal axis suitable for insertion into a body lumen, the conduit having a proximal end and a distal end;
at least one delivery waveguide and at least one collection waveguide integrated with the flexible conduit;
a transmission output of the at least one delivery waveguide and a transmission input of the at least one collection waveguide;
at least one radiation source connected to a transmission input of the at least one delivery waveguide, the radiation source constructed and arranged to provide radiation at a wavelength in a range of about 250 to 2500 nanometers;
at least one optical detector connected to a transmission output of the at least one collection waveguide;
a controller; and
a flexible, expandable first surface surrounding a segment of the conduit, the transmission output of the at least one delivery waveguide and the transmission input of the at least one collection waveguide located within said flexible, expandable first surface, said at least one transmission input movably coupled to said first surface.

38. The system of claim 37 wherein the transmission output of the at least one collection waveguide is connected to a spectrometer, the spectrometer constructed and arranged to scan radiation and perform spectroscopy at the wavelength in the range of about 250 nm to 2500 nm.

39. The system of claim 38 wherein the spectrometer and controller are configured to perform one or more spectroscopic methods including at least one of fluorescence, light scatter, optical coherence reflectometry, optical coherence tomography, speckle correlometry, Raman, and diffuse reflectance spectroscopy.

40. The system of claim 38 wherein the system is configured to scan radiation and perform spectroscopy at a wavelength within the range of about 750 nm to 2500 nm.

41. The system of claim 38 wherein the system is configured to scan radiation and perform spectroscopy using one or more ranges of wavelengths.

42. The system of claim 38 wherein the system is configured to scan radiation and perform spectroscopy using one or more discrete wavelengths.

43. The system of claim 38 wherein the system is configured to identify one or more characteristics of targeted tissue including at least one of: presence of chemical components, tissue morphological structures, water content, blood content, temperature, pH, and color.

44. The system of claim 43 wherein said one or more characteristics includes the presence of a gap between said first surface and said targeted tissue.

45. The system of claim 44 wherein the system is configured for determining the level of apposition of said first surface against adjacent tissue based on the identification of blood adjacent said first surface.

46. The system of claim 43 wherein said one or more characteristics includes a distance between said first surface and said targeted tissue.

47. The system of claim 46 wherein the system is configured for controlling the level of expansion of said first surface based on said distance of said first surface in relation to said targeted tissue.

48. The system of claim 46 wherein the system is configured for the identification of blood by inducing and detecting fluorescence.

49. The system of claim 48 comprising a dichroic filter arranged to separate radiation of wavelengths selected for delivery and radiation of wavelengths selected for collection.

50. The system of claim 48 wherein said radiation source is configured to supply radiation including a wavelength of 450 nanometers and wherein the optical detector is configured and arranged to detect a fluorescence radiation including a wavelength of 520 nanometers.

51. The system of claim 43 wherein said radiation source is configured to supply radiation of one or more wavelengths including about 532 nanometers, 407 nanometers, and between about 800 and 1000 nanometers.

52. The system of claim 51 wherein said one or more wavelengths consists of two wavelengths including at least one of about 532 nanometers.

53. The system of claim 51 wherein said system is programmed to calculate a ratio of absorbance data from the collection of said one or more wavelengths and compare the ratio with predetermined data including relationships between pre-calculated ratios of corresponding absorbance data in relation to known blood depths proximal to a vessel wall.

54. The system of claim 43 wherein the system comprises an indicator of signal intensity to an operator in relation to the identification of one or more characteristics of targeted tissue.

55. The system of claim 43 wherein the system is configured to discriminate between tissue characteristics and non-relevant artifacts including elements of the catheter and other elements artificially introduced into the body lumen.

56. The system of claim 38 wherein the system is configured to identify whether said first surface is fully expanded.

57. The system of claim 56 wherein said system is configured and programmed to identify whether said first surface is fully expanded by analyzing the characteristics of signals substantially transmitted within the circumference of said first surface.

58. The system of claim 57 wherein the signals substantially transmitted within the circumference of said first surface are directed between a plurality of transmission inputs and outputs positioned along the circumference of said first surface.

59. The system of claim 57 wherein the signals substantially transmitted within the circumference of said first surface are directed between one or more transmission inputs and outputs positioned along the circumference of said first surface and one or more transmission inputs or outputs positioned contiguously along the said flexible conduit.

60. The system of claim 56 wherein said system is programmed to analyze and compare said signals for the amount of balloon inflation media present across the path of said signals.

61. The system of claim 60 wherein analyzing and comparing signals for the amount of balloon inflation media detected comprises comparing signals transmitted between different pairs of transmission inputs and outputs.

62. The system of claim 60 wherein the programming to analyze and compare said signals compares and distinguishes signals traveling across circumferential regions about said flexible conduit.

63. The system of claim 62 wherein said circumferential regions comprise quadrants about said flexible conduit.

64-111. (canceled)
